(19) United States
(12) Patent Application Publication
(10) Pub. No.: US 2012/0046570 A1
(43) Pub. Date: Feb. 23, 2012

(54) Methods and devices for real time monitoring of collagen content and for altering collagen status

(75) Inventors: Diana Villegas, Alameda, CA (US); Abdul Tayeb, San Leandro, CA (US); Randal Jordheim, Dublin, CA (US); Martin P. Debreczeny, Danville, CA (US)

(73) Assignee: Alpha Orthopaedics, Inc., Oakland, CA (US)

(21) Appl. No.: 12/932,928

(22) Filed: Mar. 8, 2011

Related U.S. Application Data
(60) Provisional application No. 61/339,786, filed on Mar. 8, 2010.

(51) Int. Cl. A61B 5/053

(52) U.S. Cl. 600/547

(57) ABSTRACT

The present invention comprises methods and systems/devices for non-invasively measuring and/or altering collagen structures before, during and after treatment, e.g., by the application of RF energy, of tissues that comprise such collagen structures.

### CROSS-REFERENCE TO RELATED APPLICATIONS

This application claims the benefit of U.S. Provisional Application No. 60/339,786 filed Mar. 8, 2010, entitled “METHODS AND DEVICES FOR REAL TIME MONITORING OF COLLAGEN CONTENT AND FOR ALTERING COLLAGEN STATUS.” This prior application is hereby incorporated by reference in its entirety for all purposes.

### FIELD OF THE INVENTION

The current invention relates to the field of medical technology. More specifically, the present invention provides methods and devices/systems for real time monitoring of tissue treatment as well as methods and devices/systems for treatments to alter tissue content, such as collagen-containing structures, in a subject.

### BACKGROUND OF THE INVENTION

Therapeutic interventions via extreme temperature variations (cold/heat) as well as via mechanical trauma to tissue such as that provided by shockwave, ultrasound and the like, have been used in the medical field for a number of purposes. Light, ultrasound, radiofrequency, shockwave, magnetic fields and other forms of energy have been used to modify tissular temperatures and/or induce tissular disruptive mechanical trauma in order to achieve benefits such as stimulation of different biological responses, tissue contraction, ablation of tissue, or induction of necrosis. In fact, there are numerous therapeutic modalities that take advantage of such treatments. For instance, collagen stimulation, collagen denaturation and collagen contraction for the treatment of muscle-skeletal conditions, disruption of fat cells and collagen contraction for aesthetic purposes, and the reduction in size and activity of benign or malignant tumors are all medical procedures that can be achieved by temperature variation.

Thermal Treatment of Collagen

The shrinkage (or partial denaturation) of collagen molecules can be modeled as a first-order chemical kinetic process, with the specific reaction rates related to temperature via the Arrhenius relation or absolute reaction rate theory. As such, the integrated combination of exposure time and temperatures achieved results in shrinkage of collagen molecules. The activation energy required for denaturation may vary across body sites, from person to person, and at different stages of a treatment based on multiple variables. These variables include ones specific to the mechanical state of collagen, such as directionality of fiber orientation and tensile load, as well as variables associated with the chemical composition and molecular structure, such as cross-linking, that are known to vary with age, race, tissular damage, sun exposure, and smoking. Among these variables, aging has been specifically linked to increased activation energy for collagen denaturation, probably due to the age-related increases in the ratio of heat-stable to heat-labile cross-links. pH of the surrounding tissue medium and hydration status of the tissues are known to affect the activation energy for collagen denaturation as well. Thus, collagen denaturation via heating for therapeutic/cosmetic purposes is a complex process, and its ultimate success can be dependent on multiple variables such as age and past patient activity that are sometimes difficult for clinicians to measure or control.

Radiofrequency-Based Medical Treatment Devices

Radiofrequency (RF), as well as other sources of energy are capable of therapeutic effects by a well-known mechanism of action that has been well described elsewhere. RF was first introduced to the field of neurology in the 19^{th} century and since then its applications in medicine have broadened to fields ranging from neurosurgery and orthopedics to ophthalmology and surgery of the gastrointestinal tract.

One of the key shortcomings of currently (traditional) available RF technology for treating tissue is the edge effect phenomenon. In general, when RF energy is being applied or delivered to tissue through an electrode which is in contact with that tissue, the current patterns concentrate around the edges of the electrode; sharp edges in particular. This effect is generally known as the edge effect. In the case of a circular disc electrode, the effect manifests as a higher current density around the perimeter of that circular disc and a relatively low current density in the center. For a square shaped electrode there is a high current density around the entire perimeter and an even higher current density at the corners where there is a sharp edge.

Edge effects cause problems in treating tissue for several reasons. First they result in a non-uniform thermal effect over the electrode surface. In electrosurgical applications for cutting tissue, there typically is a point type applicator designed with the goal of getting a hot spot at that point for cutting or even coagulating tissue. However, this point design is undesirable for creating a reasonably gentle thermal effect over a large surface area. What is needed for a gentle thermal effect is an electrode design to deliver uniform thermal energy to the targeted tissue while avoiding the aforementioned edge and corner effects.

Energy-Based Orthopedic Treatments

In orthopedics, one of the most common objectives of energy-based modalities is the induction of the wound healing response via collagen molecule breakdown and the deactivation of C-Fibers for an antinociceptive effect. Radiofrequency (RF) energy specifically has been used to generate shrinkage of elongated joint structures, such as capsules, in a procedure known as Thermal Capsulorrhaphy, and to shrink ligaments to increase joint stability. Some of these procedures are now recognized and reimbursed by health provider organizations; however, it remains experimental in other joints, including but not limited to the ankle. In aesthetics, one of the most common objectives is the contraction of collagen as a mechanism to achieve wrinkle reduction and face/body contouring.

The early adoption of invasive RF treatment brought about by the success with thermal capsulorrhaphy led to disappointment due to the number of cases in which targeted and untargeted tissues were damaged by overheating that resulted in an unacceptable number of adverse events. The inability to know when the therapeutic objective was reached led many physicians to believe that more was better, unknowingly passing the narrow window in which hydrogen bonds are ruptured and where mechanical properties of tissue are preserved. Additional heating above the threshold for rupture of intra-molecular bonds led to breakdown of intermolecular covalent bonds with a significant decrease in tensile stiffness and a serious deleterious impact in mechanical properties of tissue. The lack of control of energy delivered resulted in significant variability of outcomes as well as the number of treatment complications. As a result, the very significant clinical benefits of thermal shrinkage of tissues were lost as a therapeutic modality. For example, Burns, J. Aesthetic Surgery Journal Nov. 1, 2005 vol. 25, no. 6:638-642, when referring to the process of skin tightening, stated that “results in any particular group are unpredictable” suggesting the need to improve patient selection as way to decrease the number of unsatisfactory outcomes.

Historically, it has previously been difficult to determine the changes in collagen-based structures in real-time and in a cost-effective way. Outcomes of a given therapeutic intervention have typically been “predicted” by indirect measurements or estimations that are clearly unreliable. The following are the most common current practices for predicting outcomes of collagen treatment and the pitfalls of their usage:

Clinical Endpoint: The most common surrogate estimation of clinical outcome during energy-based therapeutic interventions continues to be the so-called “clinical endpoints.” The primary basis for this assessment is often simply visual or tactile assessment by the clinician. Unfortunately, these physical signs are frequently only temporary responses of the tissue, such as vasodilatation resulting in hyperemia, or edema resulting from intracellular content reaching the interstitial space, and are not indicative of long-term outcome. Thus, immediate physical signs related to tissue contraction are, only to a limited extent, the result of collagen denaturation.

Impedance: Traditional tissular impedance as a determining factor of the extent of a therapeutic intervention can also be unreliable. Traditional impedance of the treated tissue can be at best difficult to distinguish from surrounding tissues and from the bulk impedance of the tissue between the area treated and the return electrode. Moreover, changes in temperature, hydration and chemical milieu of the area undergoing treatment change tissular impedance without a direct correlation with therapeutic effects.

Temperature: Temperatures achieved at the treated level are the result of multi-factorial events such as temperature of irrigating solutions, room temperature, cooling systems applied to the area treated, and ability of the tissue to remove heat by vasodilatation, among others. Even when temperature sensors are implanted within the treated tissue, the temperatures measured at those tissues are not a direct reflection of a therapeutic impact. In fact, wide ranges of temperatures at which collagen denatures have been cited by multiple authors and there is no thermodynamically defined temperature for the denaturation of collagen. In addition, denaturation is an integrated effect of both temperature and time.

Other Methods: Measured tissular contraction by MRI, ultrasound or secondary harmonic imaging microscopy may have some potential on areas of dense regularly oriented connective tissues (e.g., ligaments and tendons), since shrinkage occurs along an axis parallel to the dominant direction of fiber orientation, accompanied by swelling on the transverse axis. However, in irregularly oriented connective tissues (e.g., skin), shrinkage still occurs along the long axis of the collagen fibers, but because the fiber orientation is multidirectional in these tissues, the extent of contraction and principal axis of shrinkage is unpredictable.

There are at least two distinct risks of not knowing the actual impact of a treatment in tissue. First, the risk of under-treatment accounts for the most common situation and explains the number of insufficient treatments; under-treatment limits successful clinical outcomes. The second risk, over-treatment, is even more significant. Collagen contraction as a function of temperature is highly non-linear; the vast majority of contraction occurs in what is referred to as a “rapid transition regime,” which is as narrow as two degrees. Below these temperatures, little or no contraction is achieved, while above these temperatures, damage may ensue. In the lab, overheated specimens exhibit a time-dependent, partial recovery when returned to baseline temperatures. Over-aggressive heating can lead to tissular ablation and necrosis. Temperature-induced collagen contraction is irreversible, and the mechanical (tensile) strength of collagen decreases in proportion to the extent of contraction. When the desired outcome is collagen shrinkage, a trade-off must therefore be made between these two effects, with the ideal outcome generally being moderate shrinkage accompanied by a moderate decrease in tensile strength.

Thermal Treatment of Pain

Complaints of pain are the leading reasons for visits to doctors' offices. Among all the causes of pain, chronic back pain ranks among the most common patient complaints and is the leading cause of disability in the industrialized world; in fact, chronic spinal pain is the most rendered diagnosis by pain specialists. Non-specific back pain is reported to arise primarily from the intervertebral disc, with facet joints and sacroiliac joints following closely. Similarly, chronic cervicalgia resulting from the facet joints commonly impairs function and productivity in the workplace. When causative treatment is not feasible, it is still the physician's duty to try to ease the patient's pain. Before invasive techniques are employed, non-invasive therapies should be attempted. Medical and surgical treatments remain costly with limited efficacy. The field of interventional pain has grown considerably, and new treatment alternatives are developed; nonetheless, currently there is no gold standard for the treatment of chronic back pain.

Radiofrequency (RF) is one of several techniques utilized for neurolysis of the medial branches of the posterior primary division of the spinal nerves (paravertebral facet joint nerves). Two modalities of minimally invasive RF are currently used by pain management experts: pulsed RF (pRF) and continuous RF (cRF).

During cRF the objective is temperature-based. High temperatures are sought to ensure complete ablation of the targeted nerves to impede nociceptive output. This is achieved by placing an electrode at the neural structure and generating a destructive thermal lesion. This technique is essentially ablative. Within ablative methods, cRF is considered to have a lower incidence of long term adverse sequela. Possible complications of cRF include vascular injury, neural injury of non-targeted structures (e.g., postoperative pain, cutaneous numbness, dysesthesia, neuritis, etc.) and local infection.

Treatment of the Dorsal Root Ganglion (DRG) with cRF is an alternative to surgical rhizotomy. Its use is based on the principle that nociceptive input at the level of the primary sensory neuron could be reduced by coagulation of a small part of the DRG without causing sensory deficit. One prospective and six retrospective studies have reported beneficial effects of cRF treatment of the DRG.

During pRF the objective is electric field-based, and its impact is minimally or not neuro-destructive. In pRF, short bursts of radiofrequency energy (about 20 ms) are applied to nervous tissue followed by an “off” phase (of about 480 ms) to allow the heat to dissipate from the tissue. The tissue surrounding the electrode is exposed to the RF electric field, which induces biological effects as has been demonstrated both in cells in a cell culture and in the exposure to RF of dorsal root ganglia, resulting in transsynaptal induction of early gene expression in the dorsal horn. Initial clinical investigations have shown that pRF can be used safely as an alternative to heat lesions in patients suffering from refractory pain.

The pRF's mechanism of action has been described as neuromodulation and works by modifying the behavior of nervous tissue. The treatment is based on the principle that when a nerve is constantly subjected to painful stimuli, that nerve (through molecular processes) becomes adapted to and becomes more efficient at transmitting pain signals to the brain, thus a patient experiences more pain. pRF applies an electromagnetic field (not neuro-destructive) to restore the nerve to its original state, before it “learned” to transmit pain more efficiently. This process is called neuromodulation.

Cahana et al. (2006) reviewed current clinical and laboratory data associated with pRF use in general. The final analysis included 58 reports on the clinical use of pRF in different applications (33 full publications and 25 abstracts) and 6 six basic science reports (5 full publications and 1 abstract) and concluded that: the use of pRF is generating an increasing interest from pain physicians in the management of a variety of pain syndromes; even though the mechanism of action has not been completely elucidated, laboratory reports suggest a genuine neurobiological phenomenon altering the pain signals (neuromodulation); no side effects related to the pRF technique have been reported thus far; and further research in the clinical and biological effects is justified

Vallejo, et al. (2006) conducted a prospective cohort study on the use of pRF for the treatment of pain of the sacroiliac joint unresponsive to other forms of therapy and found that denervation with pRF of the lateral branch of the medial branch of L4, posterior primary rami of L5, and lateral branches S1 and S2 is an effective treatment for some patients with recalcitrant pain of the sacroiliac joint. Vatansever et al. (2008) found that pRF is less destructive than cRF when used on peripheral nerves. Peripheral nerves have been treated successfully with pRF. Even peripheral painful trigger points have been treated with good results. Overall, clinical results with pRF are encouraging thus far. Other basic science considerations on pRF have been established as follows: when pRF is applied to the DRG of a rat, c-fos is expressed in lamina I and II of the corresponding part of the dorsal horn; and a positive transient modulation of excitatory synaptic transmission in hippocampal organotypic nervous tissue is present.

Pulsed RF as generated by current methods is hampered by the heat generated at the electrode edge. This results from the need of having the electrode next to the targeted neural structure, and limits the amount of time and voltage that can be delivered to the target.

While thermotherapy and related treatment regimes have the potential for wide ranging application, they also, however, have the disadvantage in that it is difficult to track and control their progress. In other words, the extent of desired tissue modification (e.g., collagen denaturation of a specific area) and the extent of undesired tissue modification (e.g., damage to adjacent tissues, etc.) have been difficult or impossible to track or control even in applications where there is direct visualization of the tissues being treated. Furthermore, lack of real time monitoring of such modifications is even more problematic.

Thus, there is a continuing need for better and more controllable methods and systems/devices to allow monitoring, especially real time monitoring, of tissue treatments such as thermotherapy as well as better methods and systems/devices for more controllable treatments. The current invention provides these and other benefits which will be apparent upon examination.

### SUMMARY OF THE INVENTION

The various embodiments herein comprise multiple methods (and systems/devices to implement such) of monitoring the status of and/or treating collagen and collagen comprising tissues. In various embodiments, such monitoring can include optical monitoring (e.g., by measuring reflected light) or by electrical monitoring (e.g., by measuring electrical permittivity). The monitoring can be done before, during, and/or after a treatment to the collagen or tissue (e.g., RF treatment, application of a cosmeceutical, etc.). Also, as explained further below, the different embodiments of monitoring can optionally be used in conjunction (i.e., to monitor) with various types of treatment options performed on the collagen/tissue (e.g., RF application, heat therapy, etc.). As explained in more herein, the invention also comprises embodiments focusing on treatment of collagen fibers (e.g., through RF application) as well as numerous embodiments drawn to particular aspects of treatments. Additionally, it will be appreciated in review of this specification that various components of the system/devices can be “shared” between embodiments. For example, computer components can optionally be present in any of the monitoring/treatment embodiments and, in some instances, the same computer component can be present in more than one monitoring/treatment system or device (e.g., a single computer used to control RF treatment electrosurgery and also to monitor tissue/collagen via optical tracking, etc.).

In various embodiments herein, the invention comprises methods of monitoring a change in one or more structures (e.g., collagen) in a tissue (e.g., skin, a capsule, a vascular wall, a vaginal or urethral wall, etc.) through exposing the tissue and thus, the structure(s) to light and measuring the light reflected from one or more structures in the tissue or exposing the tissue to electricity and measuring its electrical permittivity; exposing the structures to treatment which could putatively alter them (e.g., by denaturing them); exposing the treated structures to light again and measuring the light reflected from the treated structures or exposing them to electricity again and measuring their permittivity; and comparing the light reflected from the structures or the permittivity before treatment and the light reflected from the structures or permittivity after treatment. In particular embodiments, the tissue can comprise a first and a second structure (e.g., an overlaying structure such as dermal collagen, mucosal collagen, synovial collagen, etc. and an underlying or deeper structure such as a tendon, a ligament, a fascia, or an aponeurosis, etc.). In the various embodiments, the different structures can be monitored simultaneously or sequentially or only one of the structures can be monitored.

In some embodiments herein, the invention comprises methods to utilize optical monitoring or permittivity monitoring, e.g., to establish a base line and/or to track effectiveness of treatments, etc.

In the various embodiments herein, the biological structure(s) being monitored can comprise collagen structures. The structures can be one or more of: dermal collagen, mucosal collagen, synovial collagen, a tendon, a ligament, a fascia, or an aponeourosis. In the various embodiments, the biological structure(s) can comprise, e.g., skin, a fascia, an aponeurosis, a tendon, a ligament, a capsule, a vascular wall, a vaginal wall, an introitus, or a urethra.

In some embodiments herein, the methods of monitoring collagen status and/or the methods of altering collagen status are carried out by systems/devices that include a computer processor. Such computer processor comprises an instruction set to calculate, e.g., changes in polarization or birefringence of the biological structure(s) relative to an input polarization at particular polarization angles, changes to electrical permittivity, etc. The instruction set can include instructions to determine such values relative to an input value. In the various embodiments herein, the computer processor outputs its results to a user. The output can be, e.g., in printed form, an email or text message, displayed on a screen or monitor, etc.

In certain embodiments herein, the methods can monitor the effect of any of a number of different treatments to a biological structure. Such treatments can include, but are not limited to, e.g., application of physical energy, application of radio frequency waves, application of ultrasound, application of heat, application of cold, or application of a cosmeceutical. In some embodiments, the treatment is passage of time.

In other embodiments of the methods herein, which utilize light to monitor collagen, the light to which the biological structures is exposed (and the reflected light from the structures) is polarized light (e.g., linear polarized light, circularly polarized light, etc.). In some embodiments, the light to which the structure(s) are exposed (and optionally the light reflected back from the structure) is infra-red light, UV light, light of a wave length from about 800 to about 1100 nm, or fluorescence.

In the various embodiments herein, the information on collagen structure is gathered (i.e., the collagen is monitored) without invasion of the tissue or collagen structure or with only minimal invasion of the tissue/structure, e.g., through use of a noninvasive or minimally invasive probe or shaft component of a sensor system. Such probe/shaft can be used for optical based monitoring, electricity permittivity based monitoring, etc. Furthermore, the various collagen structures can be monitored through one or more layers of untargeted tissue (e.g., overlaying tissue and/or other collagen layers).

The information gathered by the methods and devices/systems of the invention can be used to, e.g., guide clinical decisions (including decisions concerning the continuation/cessation of treatment of the tissues; the effectiveness or lack thereof of the treatments; etc.). Thus, in various embodiments, treatment can be altered, e.g., discontinued when a percent change in measured collagen change is noted through, e.g., a percent change in polarization/birefringence of the light reflected from the structure or a percent change in electrical permitivity. In other embodiments, treatment can be stopped when a certain desired result is reached in the structure being treated and/or when a certain percent change in the treated structure and/or in another ancillary structure (e.g., an overlaying dermal collagen layer) is reached. Again, such percent change is optionally indicated by a percent change in polarization/birefringence of the light reflected from the treated (or otherwise monitored) structure or in change in electrical permittivity, etc. In some embodiments herein, the methods comprise a feed-back control over treatment.

In other embodiments, the methods include the use of a plurality of source-detector distances in differentiating between changes in various collagen structures.

In some embodiments, an image of at least one tissue structure is constructed from the measurements. The image is used to assess tissue status, define a treatment area, and/or guide a course of treatment. The measurements used to construct the image can be optically or electrically based. For example, in some embodiments, measurements of optical birefringence at multiple tissue locations are used to construct an image. In other embodiments, measurements of electric permittivity collected at multiple tissue locations are used to construct an image. In some embodiments, multiple images are constructed by using source-detector separations at multiple depths. In some embodiments, multiple image portions are combined to assess tissue status or guide treatment.

In some embodiments, the methods herein for alteration of collagen and/or collagen comprising structures/tissues are carried by systems/devices that include a computer processor. For example, embodiments comprising RF treatment of collagen can be carried out through systems/devices comprising a computer processor component. In other embodiments herein, the invention comprises a system or device for monitoring a change in one or more structures in a tissue (e.g., collagen structures). Other embodiments herein comprise systems or devices to monitor change in collagen structure within cavities within a subject, while other embodiments (see below) comprise systems to apply RF energy to collagen comprising tissues. In the various embodiments, the systems can include one or more of: a light source component (configured to emit light to the tissue); one or more light polarizer components; one or more lens components; a light detection component that is configured to detect light reflected from the tissue; a lock-in amplifier component that is configured to amply the light reflected from the tissue; an electricity source component (e.g., to direct electricity into the tissue); an electricity permittivity detection component (e.g., to monitor permittivity of the tissue); and, a computer or processor component which has an instruction set that is programmed to instruct one or more of: direct the light source to expose the tissue to one or more light; direct the detection component to measure one or more reflected light from the one or more structures; and, compare the one or more reflected lights, thereby monitoring the changes in the one or more structures (based on changes in the light) and to output the results to a user (e.g., on a monitor or readout, on a printout, on a disc or other medium, etc.). Alternatively, the computer can comprise an instruction set to monitor/control application of electricity and monitoring of electrical permittivity of the tissue before, during, and/or after treatment. In many embodiments, the computer component is programmed to direct the emission and detection of the light or electricity after the tissue that comprises the structure has been exposed to a treatment (e.g., RF treatment, exposure to a cosmeceutical, application of physical energy, application of radio frequency waves, application of ultrasound, application of heat, application of cold, etc.). In some instances, the “treatment” can merely be the passage of time rather than application of a particular therapy or the like.

The computer can also be programmed to control one or more of the various components present in the various embodiments of the invention. Thus, the computer can optionally control, e.g., the intensity of the light emitted, the timing and duration of the light emitted, the degree of polarization of the light emitted to the tissue, the degree of electrical permittivity, etc. In the various embodiments, the system or device of the invention can be used to monitor tissues having a first and at least a second collagen structure. Furthermore, in such embodiments, the computer component is programmed to differentiate changes in the first collagen structure from changes in the second collagen structure. In various embodiments, the systems/devices of the invention monitor a change in one or more structures in a tissue (e.g., due to treatment of the tissue).

In various embodiments herein, the systems/devices of the invention utilized for treatment of collagen and/or collagen comprising tissues can comprise, RF energy generators (e.g., for use with electrosurgery embodiments, etc.), particular electrode designs to ameliorate or minimize “edge” or “corner” effects (e.g., for use with electrosurgery embodiments, etc.), disposable tips for use with RF treatment devices (e.g., optionally for handheld embodiments that apply RF treatments to a subject's skin, etc.), capacitive contact components (e.g., optionally for embodiments that apply RF treatments to a subject's skin, etc.), magnetic coupling sensing components (e.g., applicable to a wide range of embodiments and used to help ensure proper fittings and couplings are present in the devices used).

In particular embodiments, the systems herein can comprise systems such as (or similar to) the ones illustrated in the figures herein (and as described in the corresponding areas of the specification). It will be appreciated that various systems/devices can comprise various components. For example, some embodiments will comprise sample stages/platforms while some will not; some will utilize “handheld” monitoring devices, etc. It will be appreciated that computer/processor components can optionally control any or all of such components mentioned herein, e.g., in terms of usage and/or settings, and can optionally output any parameters set or measured for each component (e.g., light intensity, electricity levels, etc.) to a user. The various components of the systems herein are typically operably connected to at least one other component in the system of which such component is a part.

In various aspects, the invention comprises a sensor system for monitoring collagen content and/or collagen status in one or more tissues. Such sensor systems can comprise: a signal generator which generates a signal to be transmitted to one or more tissues, which signal can be corresponded to collagen content collagen status in the one or more tissues (after the signal is reflected back from the tissue or transmitted back from the tissue, etc.); a connector operably connected to the signal generator which carries the signal from the signal generator to one or more tissues and which receives one or more signals back from tissues; and a monitor which is operably connected to the connector and which generates an output corresponding to the signal back from the one or more tissues which will correspond to the collagen content or collagen status in the one or more tissues. In some such embodiments, the collagen tissue comprises a tendon, a ligament, or a capsule. In various embodiments, the systems can monitor collagen content and/or collagen status over a time period by sensing more than one signal and the signal can comprise one or more of: an optical signal, a near infrared light signal, an analog signal, a digital signal, and/or an electrical signal. In some embodiments, the system comprises a detecting probe or shaft and can comprise one or more of an indicator chemical, an optical fiber, or an electrically conductive material. The sensor systems can comprise monitors and/or signal generators that comprise a microchip(s). In some embodiments, at least part of the sensor system can be (or is capable of being) inserted into a cavity of a subject. Some embodiments include wherein the connector comprises an optical fiber and/or an electrically conductive material and/or wherein the connector is operably connected to the signal generator and/or the monitor and/or computer or microprocessor. In the various systems, the connector can carry the signal from the signal generator to the one or more tissues and carry the signal back from the one or more tissues to the monitor and/or computer or microprocessor. The connector can further comprises a shaft or probe region which can be inserted into a body cavity of a subject or into a collagen containing structure of the subject. The connector can further comprise a cable connected to the shaft or probe and/or to the signal generator and/or monitor and can optionally comprise at least one coupler. The monitors in such systems can comprise one or more displays and can optionally comprise an alarm which is triggered based on the output. Monitors can also optionally comprise one or more microchips and/or one or more computer or processing components. In the various embodiments, the sensor system can optionally convert an optical signal to a digital signal, an optical signal to an analog signal, a digital signal to an analog signal, a digital signal to an optical signal, an analog signal to an optical signal, or an analog signal to a digital signal. The sensor systems of the invention can also comprise one or more replaceable components (e.g., signal generator, connector, probe/shaft, etc.). In the embodiments, the replaceable components can comprise the connector or at least a segment of the connector, or any portion of the system that comes into direct contact with the subject. The sensor systems of the invention can optionally construct an image of the underlying tissue based on the signals detected from the tissue. Such images can comprise a display of the collagen content/arrangement/status of the tissue. In some embodiments, the system can scan across one or more tissue surfaces of a subject and a resulting image can be constructed by combining measurements at different scanned positions of the one or more tissue surfaces. The image(s) constructed can optionally be based on multi-element signals detected and can comprise a plurality of images constructed (which correspond to measurements collected with multiple source detection separations, etc.). In some embodiments, the sensor systems can comprise one or more outer portions, shells, or coverings to protect one or more components of the system. Also, the portion(s) of the system that come into contact with a subject can comprise an ellipsoid cross-sectional shape and/or a diameter of from approximately 1 mm to approximately 6 mm. Various portions of the systems can be reused multiple times and/or with multiple subjects. In some embodiments, the sensor system can compute a “grade” by combining the collagen content and/or status with subject-specific variables such as age, gender, and/or ethnicity or race. Such grade can be used to guide subject treatment or as a health parameter indicative of nutrition status and/or physical condition. Furthermore, the skin collagen content and/or status determined can optionally be used as a predictive indicator of bone collagen.

In some aspects the invention comprises methods of measuring collagen content and/or collagen status in one or more tissues in a subject by: providing a signal generator that generates a signal to be transmitted to one or more tissues and which can be related to collagen content and/or collagen status in the one or more tissues; providing a connector that is operably connected to the signal generator and which carries the signal from the signal generator to the one or more tissues and which receives one or more signals back from the one or more tissues; providing a monitor that is operably connected to the connector and/or signal generator which generates an output derived from the one or more signals back from the one or more tissues, which output corresponds to the collagen content and/or collagen status in the one or more tissues; inserting at least a portion of a signal generator and/or a connector into a cavity of the subject such that at least a portion of the signal generator and/or the connector is adjacent to a collagen containing tissue; generating a signal, transmitting it to the tissue, and receiving one or more return signals from the tissue; conveying the return signal to the monitor and/or to a computer or computer processor wherein the return signal provides information corresponding to collagen content and/or collagen status in the tissue to a user. In some such methods the subject is a human. In some of the methods, the information can be displayed as numeric information on a display of the monitor and/or computer processor and optionally can generate an alarm when collagen changes have reached a given target. In some of the embodiments, one or more component can be a replaceable component (e.g., any of the components that comes into contact with the subject). In further embodiments, the methods can also comprise removing the sensor from the subject after providing information and disconnecting the replaceable sensor component from the sensor system.

In some aspects, the invention comprises methods for monitoring a change in one or more structures in a tissue by: exposing the tissue to a first AC potential; measuring the permittivity of the exposed tissue; exposing the tissue to one or more treatments that can alter (or that putatively can alter) one or more tissular structures, thereby producing one or more treated tissues; exposing the treated tissue to a second AC potential; measuring a second permittivity of the exposed tissue; and, comparing the first permittivity and the second permittivity, thereby monitoring the change in the one or more structures. In some such embodiments, the tissue comprises a single tissular structure or layer, while in other embodiments, the tissue comprises a first and at least a second tissular structure or layer (which structures optionally can be monitored simultaneously or sequentially). When two tissular structures are present, the first tissular structure can be closer to the surface of the tissue and/or closer to the point of exposure than the second tissular structure. In the various embodiments, the tissular structure can comprise one or more of: dermal collagen, mucosal collagen, synovial collagen, a tendon, a ligament, a fascia, or an aponeourosis and the tissue can comprise one or more of: skin, a fascia, an aponeurosis, a muscle, a tendon, a ligament, a capsule, a vascular wall, a nerve, a vaginal wall, an introitus, or a urethra. Also in the various embodiments, the treatment can comprise application of physical energy, application of radio frequency waves, application of ultrasound, application of heat, application of cold, or application of a cosmeceutical. With application of various energies, the first and second AC potentials can be in the range of about 12 to about 300 volts; about 12 to about 48 volts; about 150 to about 300 volts and about 10 to 50 about mA, about 20 to 40 about mA; and/or the duration of the impulse can be between 0.05 to 10 msec. In the various embodiments, comparing the permittivities can comprise comparing AC potentials and/or comparing voltages and the first and second AC potentials can be of different magnitudes (the different magnitudes can be based on differences in the structures undergoing treatment or examination, etc.). In the various embodiments, the AC potential can be a square wave pulse, a sinusoid wave pulse, and can be controlled in its intensity and duration.

In other aspects, the invention comprises systems or devices for monitoring a change in one or more tissular structures comprising: an AC potential source component; a detection/measuring component; and, a computer component programmed which controls the AC potential; delivers a first AC potential to the analyzed tissue; directs the detection/measurement component to measure a first permittivity from the one or more tissular structures; delivers a second AC potential to the analyzed tissue; directs the detection/measurement component to measure a second permittivity from the one or more tissular structures; and, compares the first permittivity value and the second permittivity value, thereby monitoring the changes in the one or more collagen structures. In such embodiments, the tissue can comprise a single tissular structure or a first tissular structure and at least a second tissular structure. Also in some such devices, the computer component can be programmed to differentiate changes in the permittivity of the tissular structure and to control via a feed-back loop, a system providing thermotherapy or other therapeutic modality to a subject comprising the tissular structure.

In some aspects the invention comprises an electrosurgical method for noninvasively generating a wound healing response in one or more deep tissues in a subject, the method comprising: positioning an active electrode over the skin of the subject above or near-by to the one or more deep tissues; applying electromagnetic energy through the active electrode; and, providing one or more return electrodes in order to create a deep electric and thermal field sufficient to generate a thermal wound resulting in expression of at least one mediator of the wound healing response cascade. Some such embodiments can further comprise protecting the skin by controlled contact cooling (e.g., contact cooling generated from an array of thermo-electrical coolers acting over a core member active electrode to keep the core member active electrode from becoming hot). Also, some embodiments can further comprise creation of a de novo wound in one more targeted tissue (e.g., a thermal wound which optionally results in stimulation of elements of the healing response). In some embodiments of the aspect, at least one healing mediator comprises heat shock proteins or cytokines. The deep tissue can include an area of tissue that would benefit from an active wound healing response and can be one or more of, e.g., a tendon, a ligament, a fascia, an aponeurosis, a capsule, a nerve fiber, a vessel, a muscle, a bone, or other connective tissue. The various embodiments can optionally be used to treat functional ankle instability. The embodiments can optionally further comprise inducing coagulation of connective tissue and/or inducing angiogenesis in the targeted tissue. In some of the embodiments, the active electrode can be displaced to cover a volume of underlying tissue and/or the active electrode can be pulsed (e.g., pulses from about 10 msec to about 500 seconds). In some embodiments, the active electrode can be continuous).

In some aspects herein, the invention comprises an electrosurgical method for noninvasively generating electric and thermal fields in deep tissues by: positioning an active electrode over the skin of a subject above or near-by to the targeted deep tissue; applying electromagnetic energy through the active electrode; and, providing a return electrode in order to create a deep electric and thermal field sufficient to modify local biochemical milieu in nervous tissue. In some embodiments, modifying the local biochemical milieu comprises changing the expression of one or more neuropathic pain markers and/or changing the expression of one or more neuropathic pain mediators (e.g., one or more of: substance P, Glial fibrillary acidic protein (GFAP), a neurokinin-1 receptors, or Calcitonin gene related peptide (CGRP)). Modifying the local biochemical milieu can also comprise changes to the expression of mitogen-activated protein kinases (MAPK). Embodiments can comprise wherein the nervous tissue comprises C-Fibers and wherein the thermal field has an antinociceptive effect by deactivating the C-Fibers; wherein the applied electric and/or thermal fields are adjusted based on a measurement of tissue impedance; wherein the nervous tissue is poorly vascularized, thereby allowing for differential heat retention by the nervous tissue and surrounding well-vascularized tissue; wherein the thermal field induced in the deep tissues is kept below 45 C; wherein neuromodulation is induced in the nervous tissue; wherein the thermal field induced in the deep tissues is kept between 45 and 55 C; wherein neurolysis is induced in the nervous tissue (e.g., wherein unmyelinated fibers are targeted for neurolysis); and wherein the combined effects of neuromodulation and neurlysis are induced in the nervous tissue.

In other aspects the invention comprises a skirt thermoelectric cooling device which device is attached circumferentially to the periphery of an electrode to be used on one or more tissues of a subject, and which mitigates electrode edge effects when the electrode is used on a tissue. Some such devices can be attached to either a directly coupled or capacitive coupled electrode, e.g., wherein the electrode edge effect is reduced by inclusion of a transitional alloy electrode.

In other aspects, the invention comprises a temperature controlled electrode to conduct energy to one or more tissues of a subject, wherein the electrode is capable of being used in direct contact with the tissue, and wherein a surface of the electrode in contact with the one or more tissues is temperature controlled. In some such embodiments, the energy can be radiofrequency energy and energy from the electrode can be delivered in monopolar or bipolar fashion. The electrodes can optionally comprise a round or polygonal shape. In some embodiments, a cooling system can be provided the periphery of the electrode (e.g., comprising thermo-electric coolers attached to the periphery of the electrode, either parallel to the electrode surface, perpendicular to the electrode surface or at different angles to the electrode surface). In some embodiments, the area cooled by the temperature controlled electrode exceeds an area of energy transfer by the temperature controlled electrode. The electrode can comprise concentric layers of materials constructed so that thermal transfer is maximum at the edges and minimum at the center or can comprise concentric layers of materials constructed so that electrical energy transfer is maximum at the center and minimum at the edges. In either instance the different layers can be electrically isolated from each other and different radiofrequency generators can drive different levels of energy through the different layers so as to create a gradient of electrical energy transfer. In some embodiments, the electrode can comprise a variable thickness dielectric material that provides power attenuation wherein electrical energy transfer is higher at the center of the electrode and lower at the periphery of the electrode. The electrode can also comprise a cast alloy constructed so that electrical energy transfer is higher at the edges and maximum at the center. The concentric layers of the various embodiments can comprise, e.g., wherein a first layer comprises silver, a second layer comprises cooper, a third layer comprises aluminum, and a fourth layer comprises iron. Furthermore, the different layers can be comprised of electrically conductive materials with a variety of thermal conductivity properties and a variety of electrical conductive properties. In some embodiments, a cast alloy is constructed so that thermal transfer is maximum at the edges and minimum at the center, while in some embodiments variable thickness can be used to control thermal transfer. In some embodiments the electrode can further comprise a cooling system comprised of a primary subsystem and a secondary subsystem, e.g., wherein the primary and the secondary subsystems are thermally coupled through a fluid medium. In such instances, the primary subsystem can optionally precisely regulate the temperature of the electrode tip, while the secondary subsystem can optionally utilize an active system of TECs and/or releases heat to the environment through a passive system radiator and/or regulates the fluid temperature to a set point or less than or equal to a set point. In the various embodiments, the primary subsystem can comprise one or more TECs which can be electively driven to cool or heat in response to a varying RF load. The embodiments can also further comprise one or more disposable tip which maintains thermal and electrical contact between the electrode and the tissue of a subject and provides a physical barrier between the electrode and the tissue; the disposable tip can comprise an electronic chip used to track tip usage or time expired since manufacture. In some instances, the embodiments can further comprise a capacitive proximity sensor wherein a dielectrically isolated sensing electrode is placed adjacent to an RF delivery electrode on a tissue and which is able to sense an applied RF voltage and/or can further comprise a plurality of sensors wherein the members of the plurality are placed around the treatment electrode so as to detect the proximity of the perimeter of the RF delivery electrode to the tissue. In some such embodiments, the proximity of the electrode to the tissue can be provided as feedback to the user to guide electrode placement and maintain effective contact between the electrode and tissue. In some embodiments, the energy can be prevented from being conducted through the electrode when the proximity between the electrode and tissue is insufficient. Also, in some embodiments, the proximity between the plurality of sensors and the tissue can be used to guide the distribution of energy applied across the electrode surface. Embodiments can also include wherein the members of the plurality of sensors are placed around the treatment electrode so as to detect the proximity of the perimeter of the RF delivery electrode to the tissue, and wherein the RF delivery electrode is directly coupled to the tissue as well as wherein the members of the plurality of sensors are placed around the treatment electrode so as to detect the proximity of the perimeter of the RF delivery electrode to the tissue, and where the RF delivery electrode is dielectrically coupled to the tissue.

In some aspects the invention comprises a fluidics connector for connecting two or more fluid transporting conduits, which connector comprises a permanent ring magnet attached to a removable half of the connector on one fluid transporting conduit which ring magnet activates a magnetic sensor when in the proximity of the other half of the connector on the other fluid transporting conduit. In some such embodiments, the removable half of the connector can be free to rotate around its axis and still maintain detectability by the sensor; the ring magnet can be insert-molded into the removable half of the connector; the ring magnet can be attached to the removable half of the connector post manufacture; the ring magnet can be polarized axially; the ring magnet's north pole can be facing the half of the connector containing the magnetic sensor; and/or the magnet's south pole can be facing the half of the connector containing the magnetic sensor.

These and other features of the invention will become more fully apparent when the following detailed description is read in conjunction with the accompanying figures and claims.

### BRIEF DESCRIPTION OF THE DRAWINGS

FIG. 1a presents a schematic of an example configuration of optical components of a monitoring device positioned in relation to a tissue surface which can be used in conjunction with various embodiments of the invention.

FIG. 1b presents a schematic of an example configuration of electrical components of a monitoring device which can be used in conjunction with various embodiments of the invention.

FIG. 2a presents a diagram of a knee joint illustrating where collagen-containing structures to be treated/monitored are present within a cavity area.

FIG. 2b presents cross-sectional and lengthwise sections of exemplary probe or shaft components of a sensor device of the invention as well as a sketch of an exemplary optical probe component of the invention.

FIG. 2c illustrates insertion of a probe/shaft component of a sensor system into a cavity area of a subject's knee joint.

FIG. 2d presents a schematic diagram of an exemplary monitoring device that can be used with various embodiment of the invention.

FIGS. 3a-c show controlled internal heating (without surface heating) of a tissue through use of exemplary mdRF devices/methods of the invention.

FIGS. 4a-h show various views of an exemplary handheld temperature controlled electrode device of the invention.

FIGS. 4i-j show computer simulations of temperature profile on surface with use of an exemplary temperature controlled electrode of the invention.

FIG. 4k shows experimental data from use of an exemplary mdRF device of the invention.

FIGS. 5a-b present an exemplary disposable electrode tip embodiment of the invention as well as an exemplary flex circuit trace and circuit.

FIGS. 6a-c shows a schematic depiction of tissue in contact with a treatment electrode and a sensor electrode of an embodiment of the invention having a capacitive contact sensing aspect (6a), while a SPICE circuit simulation is shown in FIG. 6b and the data is extracted and plotted in FIG. 6c.

FIG. 7 illustrates an exemplary fluidics connector employing magnetic sensing, of the invention.

FIG. 8 illustrates an exemplary arrangement of optical fibers at the tissue interface in an embodiment of the invention.

FIG. 9 illustrates an exemplary arrangement of a temperature controlled electrode of the invention.

### DETAILED DESCRIPTION

The ability to accurately monitor (and thereby more accurately control) tissue treatments such as thermotherapy, especially in real time, is significant in the productive treatment of a number of disease states/medical conditions, e.g., treatment of joint trauma. Furthermore, the ability to accurately monitor the effect of such products as cosmeceuticals on tissues (whether during and/or after treatment/application of such) is quite significant. Various embodiments of the current invention utilize tracking of changes of reflected light from biological structures or changes in electrical permittivity, to track corresponding changes in such structures arising from treatment. Of course, it will be appreciated that even though the various embodiments herein are primarily described as useful for tracking progress of tissue treatment and the like, that the benefits of the invention also extend to, e.g., monitoring the condition of a subject's tissue or the presence and/or progression of a disease state or medical condition, whether or not any treatment is administered. Other embodiments of the invention present methods and systems/devices involved in the actual treatment of collagen or other tissues or tissue structures in a subject. Such treatment methods and use of such systems/devices can optionally be monitored through any of the various monitoring embodiments herein. It will be appreciated that any of the various monitoring embodiments herein can optionally be used in conjunction with any of the various treatment embodiments herein, e.g., as concurrent and/or complementary applications. In some instances, the monitoring and treatment embodiments (e.g., any combination of embodiments herein) can be integrated into a single device or system, while in other instances the monitoring and treatment embodiments can be in separate devices or systems but used together (again, e.g., concurrently, sequentially, or in a complementary fashion, etc.).

Optionally the changes in collagen are tracked in real time in the monitoring embodiments, e.g., during treatment of the tissue. In yet other embodiments, the invention uses monitoring procedures to track changes after treatment has occurred (e.g., rather than as treatment is occurring). Again, the invention includes the methods of monitoring treatment effects as well as systems and devices that implement such methods and also includes methods and systems/devices to treat or alter collagen or other tissues or tissue structures. Overall, the invention results in increased monitoring ability for tracking of tissue treatment and increased ability to effectively treat collagen and collagen comprising tissues.

It will be appreciated that various of the embodiments herein (including, but not limited to various devices/methods for monitoring collagen within cavities within a subject, devices/methods for electrical monitoring of collagen content/status; devices/methods for wound healing; temperature controlled electrodes for use in treatment (including disposable tips for such and use of contact sensing aspects to help monitor accurate electrode/tissue contact, etc.) can be used with, or in conjunction with, various embodiments for collagen treatment/monitoring found in other patent applications filed by the inventors such as U.S. Ser. No. 61/066,593 filed Feb. 20,2008; PCT/US2009/001093 filed Feb. 20, 2009; U.S. Ser. No. 12/380,014 filed Feb. 20, 2009; U.S. Ser. No. 61/274,704 filed Aug. 19, 2009; U.S. Ser. No. 12/806,811 filed Aug. 19, 2009; U.S. Ser. No. 61/339,786 filed Mar. 8, 2010.

### Definitions

Before describing the present invention in detail, it is to be understood that the invention herein is not necessarily limited to use with particular light sources, thermotherapy treatments, etc., which can, of course, vary. It is also to be understood that the terminology used herein is for the purpose of describing particular embodiments only, and is not necessarily intended to be limiting. As used in this specification and the appended claims, the singular forms “a,” “an,” and “the” include plural referents unless the context clearly dictates otherwise. Thus, for example, reference to “a sensor” optionally includes a combination of two or more sensors, and the like.

The term “subject” as used herein includes, but is not limited to, a mammal, including, e.g., a human, non-human primate (e.g., monkey), mouse, pig, cow, goat, rabbit, rat, guinea pig, hamster, horse, monkey, sheep, or other non-human mammal, or a non-mammal, including, e.g., a non-mammalian vertebrate, such as a bird, reptile, or amphibian. In some embodiments, the methods and systems/devices of the invention are used to monitor and/or treat non-human animals. Many commercially important animals are susceptible to medical conditions, e.g., joint trauma, whose treatment is optionally monitored and/or treated with embodiments of the current invention.

The phrase “pain mediators” (e.g. Substance P, NK1, NK2, CGRP) is used herein to describe substances involved in nociception, transmitting information about tissue damage from peripheral receptors to the central nervous system and converted as sensation of pain. It has been theorized that they play a part in fibromyalgia. Substance P is a protein found in the brain and spinal cord and is associated with some inflammatory processes in the joints. Its function is to cause pain, particularly in arthritis, low back pain and fibromyalgia. Release of substance P has also been associated with migraine headaches. A role of substance P and neuro-peptides such as NKA in nociception is suggested by the reduction in response thresholds to noxious stimuli by central administration of NK1 and NK2 agonists. Calcitonin gene related peptide (CGRP) is a member of the calcitonin family of peptides, which in humans exists in two forms, α-CGRP and β-CGRP. α-CGRP is a 37 amino acid peptide and is formed from the alternative splicing of the calcitonin/CGRP gene located on chromosome 11. CGRP is one of the most abundant peptides produced in both peripheral and central neurons. It is the most potent peptide vasodilator and function in the transmission of pain. NeuroKinin A (NKA) is a pain mediator neuro-peptide of the kinin group of proteins. Substance P and NK1 are closely related; they are produced from a polyprotein precursor after differential splicing of the preprotachykinin A gene. Bradikinin is a physiologically and pharmacologically active peptide of the kinin group of proteins. The B1 receptor (also called bradykinin receptor B1) is expressed only as a result of tissue injury, and is presumed to play a role in chronic pain. This receptor has been also described to play a role in inflammation. TNFα is a cytokine involved in systemic inflammation and is a member of a group of cytokines that stimulate the acute phase reaction. The primary role of TNF is in the regulation of immune cells. TNF is also able to induce apoptotic cell death, to induce inflammation, and to inhibit tumorigenesis and viral replication. Glial fibrillary acidic protein-specific antibodies (GFAP) is a filament protein expressed by cells in central nervous system.

As will be appreciated, the above terms, as well as additional terms, are detailed/described further below.

### Real-Time Monitoring of Tissue Treatment

In various embodiments of the present invention, changes in biological structures (e.g., collagen structures) are monitored by directing light into a tissue and collecting the light after it has interacted with the structures within the tissue or by directing electricity into a tissue and monitoring changes in electrical permittivity. In particular embodiments, the collected light or electrical permittivity is measured in order to monitor the status of and/or changes in the structures within the tissue. In the various embodiments herein, more than one structure can be monitored simultaneously or sequentially, e.g., during the course of a treatment process that involves the tissue or during the course of the progression of a disease state or medical condition. Again, it will be appreciated that the various embodiments herein can be used in conjunction with the inventions described in, e.g., U.S. Ser. No. 61/066,593 filed Feb. 20,2008; PCT/US2009/001093 filed Feb. 20, 2009; U.S. Ser. No. 12/380,014 filed Feb. 20, 2009; U.S. Ser. No. 61/274,704 filed Aug. 19, 2009; U.S. Ser. No. 12/806,811 filed Aug. 19, 2009; U.S. Ser. No. 61/339,786 filed Mar. 8, 2010.

Particular embodiments of the invention monitor, through exposure to light, the status and/or change in status of collagen. Collagen is a uniaxial birefringent material whose optic axis (or slow axis), in which direction light travels most slowly, is parallel to the long axis of its triple helix while its fast axis, the one in which direction light travels most quickly, is perpendicular to its triple helix axis. The difference in refractive index between the slow and fast axes of collagen is approximately 3×10⁻³. See, e.g., D. J. Maitland and J. T. Walsh, “Quantitative Measurements of Linear Birefringence During Heating of Native Collagen” Lasers in Surgery and Medicine, 20:310-318 (1997). Linearly polarized light that is oriented parallel to the collagen optic axis will undergo no polarization rotation upon interaction with the collagen. However, linearly polarized light at a 45 degree orientation will undergo maximal polarization rotation.

The birefringence of collagen is lost upon denaturation. Therefore, various embodiments herein can monitor changes in birefringence of a tissue (e.g., before and after thermotherapy) in order to track the progression of collagen denaturation by heating, etc. Thus, for example, a tendon can be monitored before treatment and the birefringence thus determined can be compared to the birefringence determined after (or during) treatment to thereby monitor changes if any in the tendon (such as denaturation of its collagen). See Maitland, supra.

Other embodiments herein monitor changes in biological structures (e.g., collagen) by monitoring changes in electrical permittivity of a tissue. Thus, in some embodiments, a tissue undergoing treatment can be monitored by having its electrical permittivity measured before treatment and again during and/or after treatment (e.g., RF treatment) to track collagen denaturation or alteration.

Various embodiments of the invention utilize the change in birefringence due to denaturation or change in permittivity to monitor, e.g., the progress or effectiveness of treatments and the like. In particular embodiments, the baseline or starting status of a structure (e.g., a collagen layer) and the structure's response to treatment are both monitored by directing, e.g., a linearly polarized laser light into the tissue which comprises the structure, collecting the light after it has interacted with the structure(s), and measuring polarization-dependent properties of the collected light (e.g., the extent or degree of depolarization, the amount of polarization rotation, etc.) or by directing electrical charges into a structure and measuring the change in electrical permittivity. In various applications, the starting status of the structure is typically the status before any treatment is applied to it. However, the starting status can also optionally be from a point after treatment has started. Thus, for example, monitoring can optionally be implemented in the middle of a course of treatment of a tissue and be used to track changes occurring after the start of monitoring.

In various embodiments, the monitoring can be done non-invasively (e.g., by directing light upon skin, by administering an electric charge on the skin, etc.), while in other embodiments, the monitoring can involve an invasive act (e.g., a monitoring probe or component inserted (e.g., arthroscopically) into a subject to monitor a tendon, vessel wall, etc.).

In some embodiments the invention provides an image of tissue structures that may be useful in defining the treatment area in addition to guiding the treatment. In some embodiments the image allows location of a deeper structure beneath a shallow structure. For example, for non-invasive treatment of a tendon lying beneath the skin, the tendon may be observable in the image, and this may help define the treatment area. Further, the evolution of the image with treatment may guide the course of treatment. In some embodiments, the image is created by scanning across the surface of the tissue and constructing an image from the scanned measurements. Optical scanning may be accomplished, for example, through the use of a mirror held on a motorized stage. In some embodiments, the detector consists of an array of separately addressable elements (e.g. CCD camera) from which the image is constructed.

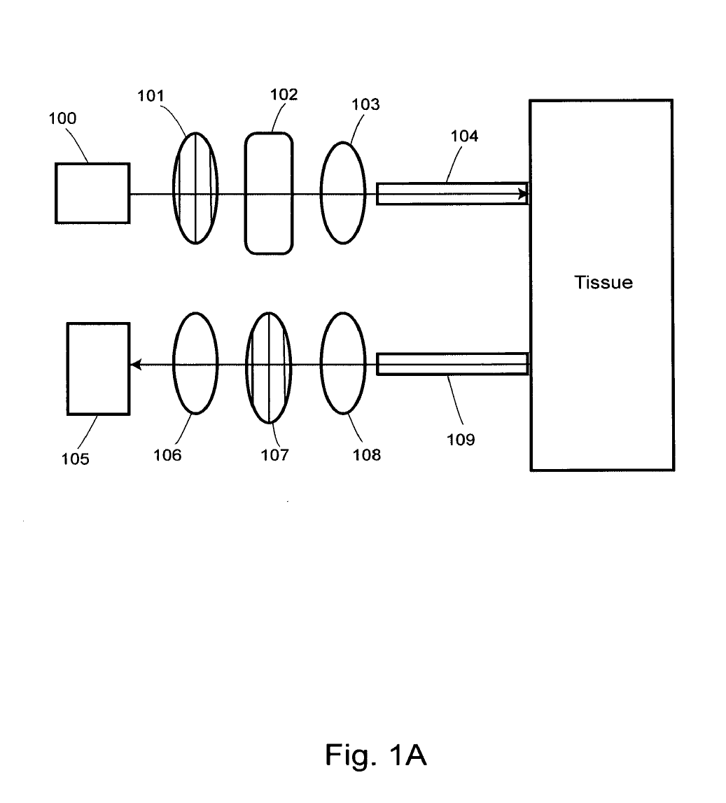

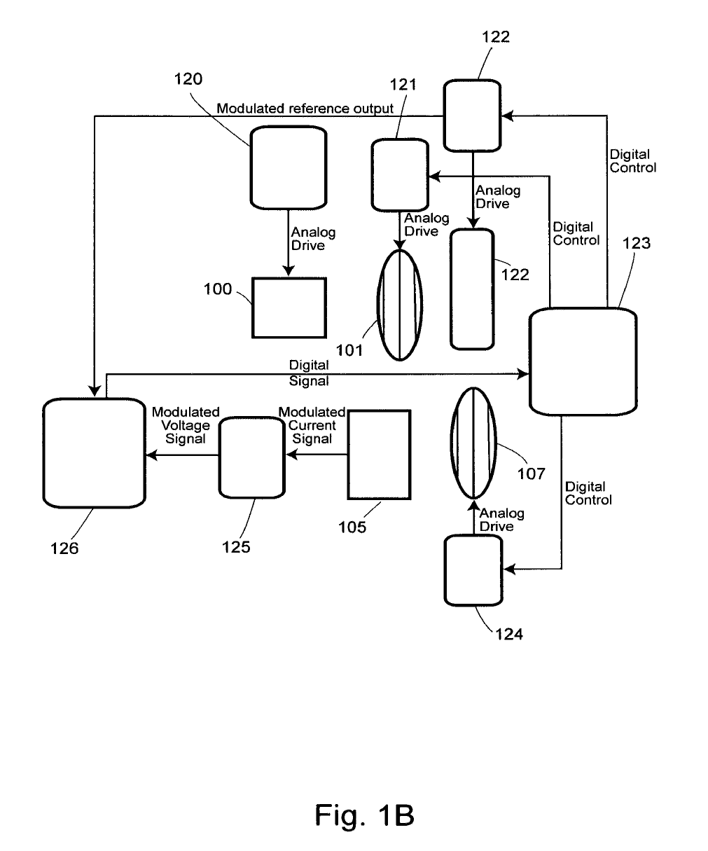

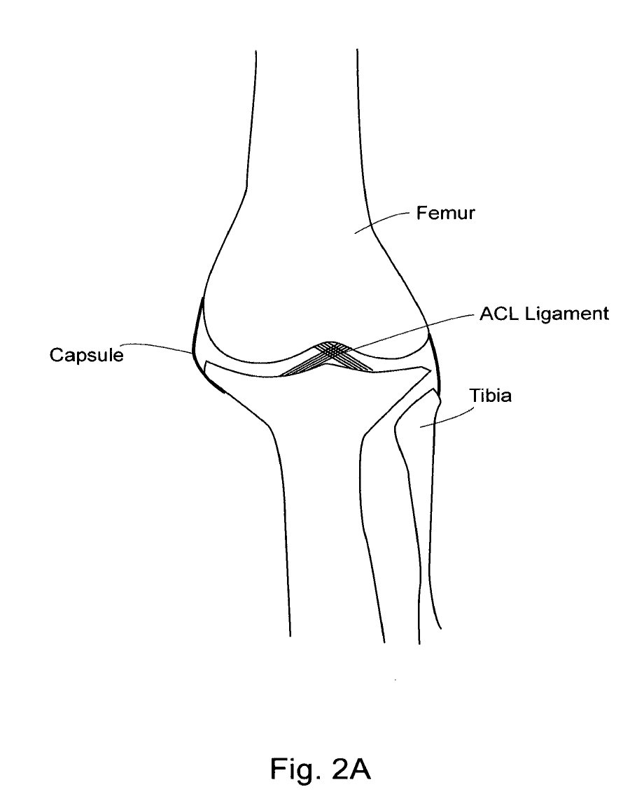

An exemplary detector for monitoring collagen content or collagen status in a tissue can be seen in FIG. 1a. In the figure, detector 105, can be a CCD detector, allowing separate birefringence measurements to be computed at each pixel location of the CCD. The displayed image can thus be a map of birefringence. Pixel elements located above a tendon will show increased birefringence compared to pixels that are not directly over the tendon. In some embodiments, the illumination and/or detection system includes an array of fiber optic elements. For example, in some embodiments, both the illumination and detection systems can comprise arrays of fiber optic elements. On the illumination side, the light source (e.g. laser) is coupled into a packed array of fiber optics. At the tissue sensing interface, the array of light source fibers can be interspersed with an array of detection fibers. The detection fibers are then bundled and imaged onto the detection system (e.g. in place of component 19 in FIG. 1a).

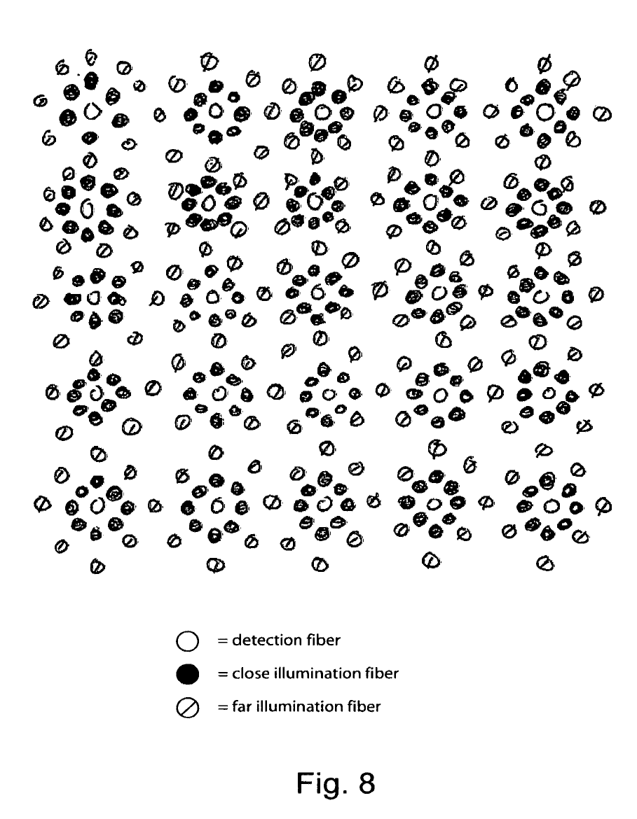

In many embodiments the detection fibers will be positioned in a well-ordered array at the tissue interface, and this ordering is preserved when forming the bundled array of fibers that is imaged onto the detector array. A minimum separation between the source and detector fibers may be maintained at the tissue interface. For example, in some embodiments, this minimum separation is a center-to-center distance of 0.5 mm. As discussed above, maintaining this minimum source-detector separation may help to increase the effective penetration depth of the degree of birefringence (DoB) measurement. In yet other embodiments, at least two source-detector separations are employed in constructing the fiber array. An example embodiment is depicted in FIG. 8. In this embodiment the two illumination fiber types (“close” and “far”) may be separately and/or alternately illuminated so that separate images can be constructed from the 2 different illumination sources. In some embodiments, the “close” image will be more provide more information on structures located near the sensor-tissue interface, while the “far” image will provide more information related to deeper-lying structures. In some embodiments information contained in the “close” image will help to guide treatment, such as to prevent damage to shallow structures, while treating deeper structures. The “far” image is used to assess status of the treatment of the deeper structures. In some embodiments, the center-to-center separation between the “close” source fibers and detection fibers is in the range of 0.1 to 2 mm, whereas the “far” source fibers are separated from the detection fibers by a distance in the range of 0.2 to 5 mm.

In some embodiments, different portions of the image(s) can be combined to provide a result indicative of collagen status or treatment extent. For example, within a single image, a portion of the image collected in a tissue region where a targeted structure is directly beneath the measurement array may be combined with a portion of the image collected peripherally to the targeted structure. As another example, portions of multiple images may be combined such as collected with multiple source-detector separations, or as collected as a function of treatment. In some embodiments, the methods of combining the multiple image portions involves addition and/or subtraction of the degree of polarization or birefringence across the multiple image elements. In other embodiments, the multiple image elements are provided as input to a mathematical model of the tissue structure, from which a property or properties of the tissue structure (e.g. birefringence of a tendon) are then derived. Many suitable mathematical models of tissue structure are known in the art, such as further described in and included her by reference Principles and advanced methods in medical imaging and image analysis, Atam P. Dhawan, H. K. Huang, Dae-Shik Kim, eds., World Scientific, 2008; Optical-Thermal Response of Laser-Irradiated Tissue, Ashley J. Welch, Springer, 2010; Handbook of Mathematical Methods in Imaging, Otmar Scherzer, Springer, 2010.

In some embodiments, in analogous fashion to the optical measurements described above, measurements of electrical permittivity are combined to form an image.

In some embodiments, the image is displayed in real-time on a monitor that can be used by the clinician to locate the treatment area, with the optional additional capability of guiding the course of treatment. In some embodiments the image tracks the amount of treatment applied to each tissue region. This can be useful, especially when multiple treatment passes are made over the same tissue area. In such cases, the image may provide an indication of cumulative treatment in each segment of the image.

In some embodiments, the invention can be used to determine collagen content in a tissue (e.g., whether or not any treatment has been or is to be administered). For example, light properties such as birefringence, permittivity, etc. can be measured in multiple subjects and/or at multiple sites within a subject to create a measurement guide of collagen content/status based on the property measured (i.e., as opposed to changes in such property used in some embodiments herein). Based on multiple readings (between the level of the property measured and collagen content), such measurement guide thus allows a practitioner to measure or estimate the collagen level in a tissue. The measure/estimate of collagen (based on the property measured) can be done prior to any treatment to the subject or to compare with an average measurement (e.g., as in comparing diseased tissue against non-diseased tissue, etc.). Thus, a practitioner can use such measurement to advise whether treatment should even be undertaken, whether or to what extent treatment may be successful, etc. The readings taken to construct the measurement guide can optionally be normalized for subject status (e.g., based on age, ethnicity, gender, etc.) and tissue type or location (e.g., dermal collagen in the face, dermal collagen in the hands, etc.).

It will be appreciated, that while for ease of description, the current description herein primarily describes the structure type that is monitored as collagen, other structures are optionally included in the various embodiments. Thus, it is contemplated that the invention can also find use with monitoring of, e.g., pathological tissue such as tumors that are treated with thermotherapy. In such embodiments, it is thought that the methods and systems of the invention will track such tissues via, e.g., birefringence, other optical methods such as fluorescence, or electrical permittivity. Furthermore, in some embodiments the structure monitored can comprise keratin and/or elastin.

### Exemplary Uses of the Methods/Devices

In various embodiments, the methods of the invention comprise placement and orientation of the various system components (e.g., light emitter and detector) in relation to the tissue/structure being monitored and/or treated. As will be appreciated, such placement/orientation can involve movement of the tissue being monitored or treated and/or movement of one or more components of the devices/systems herein. Also, while the various embodiments herein are primarily discussed in terms of generalized systems of components, particular monitoring and/or treatment embodiments can comprise self-contained devices (e.g., a handheld device and/or a handheld device operationally connected to a unit having computer components, etc.). Furthermore, as mentioned throughout, the various methods of the invention and the various devices/systems of the invention can be used topically on subjects (i.e., noninvasively) and/or can be used internally within subjects (i.e., invasively either through incisions or the like or through orifices of the subjects) through use, or along with use of, probes/shafts such as detailed herein.

By determining a baseline status measurement of a tissue structure's birefringence or permittivity, various embodiments of the invention allow comparison of such values with measurements taken after/during a treatment (or even taken at a later date) to establish the impact (if any) of a given intervention. Thus, the monitoring can be real time during the treatment and/or after the treatment. Additionally, in some embodiments by simultaneously monitoring collagen at multiple settings while delivering a treatment (e.g. radiofrequency, ultrasound, light) the treatment can be terminated when either the target structure (e.g., a tendon) or a non-target or secondary target structure (e.g., a superficial layer such as dermal, synovial, mucosal collagen, etc.) has reached the desired change or has exceeded a threshold change, in the particular structure. Thus, through use of some embodiments, a particular collagen containing structure can be treated while simultaneously avoiding damage to other collagen containing structures located above or below the targeted structure.

### Monitoring and Treatment System Overviews

The description of various embodiments of the systems/devices of the invention and their uses herein presents the basic components of the embodiments in a number of exemplary monitoring and treatment arrangements. In various such monitoring and treatment illustrations, the embodiment is described as arranged to monitor (e.g., as for tracking progress of a treatment, e.g., via RF treatment) a tissue structure such as collagen in a tendon. Of course, it will be appreciated that the various component arrangements in the embodiments should not necessarily be taken as limiting. Finally, while the various embodiments present several devices/components and arrangements, it will be appreciated that not all such features or arrangements are necessarily present in all embodiments unless specifically stated.

Again, the various embodiments herein can optionally be used in conjunction with the embodiments described in U.S. Ser. No. 61/066,593 filed Feb. 20,2008; PCT/US2009/001093 filed Feb. 20, 2009; U.S. Ser. No. 12/380,014 filed Feb. 20, 2009; U.S. Ser. No. 61/274,704 filed Aug. 19, 2009; U.S. Ser. No. 12/806,811 filed Aug. 19, 2009; U.S. Ser. No. 61/339,786 filed Mar. 8, 2010.

FIG. 1a shows a schematic that outlines the basic optical components that are found in a number of exemplary embodiments of the invention which utilize optical monitoring embodiments. As can be seen in FIG. 1a, light source 100 (e.g., a miniature laser such as a vertical cavity surface emitting laser) emits light which travels through excitation polarizer 101 and then through polarization rotator 102 and focusing lens 103. The light then traverses optional polarization preserving fiber 104 and enters into a tissue layer. Although only a single fiber is depicted, in some embodiments, for example for imaging purposes, or in order to spread the measurement over a wider area, a multiplicity of fibers may be used. See above. As will be appreciated, depending upon the tissue type, the structures present in the tissue, the strength of the light, etc., the light can penetrate to various depths within the tissue. Once within the tissue, the light is reflected from various structures, exits back out of the tissue and is captured by optional polarization preserving fiber 109. As with the illumination side, the fiber on the detection side can optionally include a multiplicity of fibers. Again, see above. The light further passes through collimating lens 108, detection polarizer 107, detection lens 106 and into detector 105.

An overview of several basic electrical components present in various embodiments utilizing optical monitoring of collagen state is shown in FIG. 1b. In FIG. 1b, computer 123 provides digital control signals for polarizer controllers 121 and 124 and polarization rotator controller 122 which modulates the polarization between two linear polarization states. A current source (e.g., source 120) can be used to power light source 100. The current source is optionally a direct current power source. The polarization rotator controller can also provide a modulation signal used for lock-in amplification (LIA) of signals (by lock in amplifier 126 and trans-impedance amplifier 125) detected by detector 105. The lock-in amplification provides a digital signal that is read in by the computer and reported back to the user. Use of and/or control of the various components as shown in FIGS. 1a and 1b can be guided by a computer software algorithm.

As stated previously, not all embodiments will necessarily comprise all elements/components listed herein or described in particular figures. For example, some embodiments that use optical monitoring do not include a lock-in amplifier. Embodiments concerned with probe monitoring via optical changes in cavities within a subject may not include methods/devices directed to electrical monitoring of collagen changes or temperature controlled electrodes, etc. Correspondingly, it will be appreciated that other embodiments comprise additional components than those shown in particular Figures. For example, various embodiments for optical monitoring of collagen status can comprise polarization compensators, split sampling lenses, etc.

As explained throughout, other embodiments of the invention comprising monitoring of collagen treatment, etc. via tracking of electrical permittivity. Such devices/systems can optionally comprise similar components in many instances as the optical monitoring configurations. For example, power sources, computer units, optional probes/shafts, etc. can be present in permittivity embodiments, as well as components directed to generation of controllable electrical charges to be sent through the tissue under observation and detectors to measure the permittivity in such tissue.

In the various treatment embodiments herein, the systems/devices can comprise a number of components such as RF generators to produce the energy used to treat the tissues, and computer components, etc. used to help control the processes as well as particular components such as disposable electrode tips, capacitive contact sensors, and particular electrode designs used to help control skin surface temperatures. See below.

It will also be appreciated that any of the various connections in the different embodiments herein (whether monitoring embodiments or treatment embodiments) is optionally analog or digital. Digital signals are convenient for long-distance corruption-free transmission of signals, while analog signals are more often used over shorter distance to accomplish the direct interface with mechanical or optical components.

Although optical fibers provide a convenient way of transporting light into a device, various “free-space” embodiments of the systems are also included in the invention. Such optional free space systems comprise the benefit of avoiding the inevitable losses associated with coupling light into optical fibers. However, other embodiments can optionally include optical fibers (e.g., those monitoring embodiments used for internal monitoring). See below.

Additional components and arrangements of components in the systems of the invention are described throughout.

Light Sources

In some embodiments herein, the monitoring of tissue (e.g., as in tissue treatment through change in particular biological structures) is accomplished by light excitation and camera observation. As described below, other embodiments of the invention monitor tissue via electrical permittivity. In the embodiments that utilize light sources, it will be appreciated that the invention is not necessarily limited by particular type or specific example of illumination used. Thus, in various embodiments, the light source can comprise, e.g., a laser, an edge-emitting laser diode (e.g., as opposed to a vertical cavity emitting laser diode, VCSEL), a resonant cavity LED, a gas laser (e.g. HeNe), a Nd-YAG laser (e.g., 1064, 532, or 355 nm), an YLF laser (e.g., 1053), a non-laser excitation source such as an LED, halogen or xenon arc lamp, etc. In the various embodiments, the light source utilized can comprise a monochromatic light of a wavelength that allows for deep penetration into tissue. Those of skill in the art will be familiar with numerous light emission devices that can be used in conjunction with the various embodiments of the invention. Depending upon, e.g., the type of tissue to be monitored, whether the monitoring is done topically or arthroscopically, etc., the intensity/power of the light can be correspondingly chosen or adjusted. The optical properties of tissue have been well characterized (see, e.g. “Optical-Thermal Response of Laser Irradiated Tissues,” ed. A. J. Welch, M. van Gemert, Springer, 1995) and many theoretical models have been developed to estimate tissue penetration (see, e.g. “Photon Migration in Tissues,” ed. Britton Chance, Springer, 1989). Furthermore, the penetration depth of detected photons can also depend on the source-detector spacing as discussed further herein. Various embodiments can utilize IJV dichroism and thus have a range of wavelengths. Suitable sources can include: (1) a plurality of discrete wavelength sources, (2) tunable sources, and (3) broadband sources whose wavelength region is selected or tuned by a secondary mechanism, such as an optical filter. As an alternative to the use of linearly polarized light, circularly polarized light can also be employed in the embodiments herein.

Lenses

In the various embodiments herein directed towards optical monitoring, a number of different lenses and lens types can optionally be used. For example, split sampling lenses, can be used in some embodiments herein. Furthermore, various embodiments herein can comprise lenses that are not split sampling lenses. Those of skill in the art will be exceedingly familiar with selection and orientation of lenses suitable for use with the various light sources used in the embodiments herein.

Polarizers

In various embodiments herein utilizing optical monitoring, the light used to monitor the tissue can be generally substantially linearly polarized from an emitting device (e.g., a laser). However, particular embodiments can also optionally include a polarizer to increase the polarization extinction ratio by rotating the polarizer for maximum transmission of the light source. While the rotation of the polarization can be accomplished by a number of ways, certain embodiments rotate the polarizers and/or polarization rotator either manually or mechanically. Thus, in particular embodiments herein, a polarizer and/or a polarization rotator in a suitable housing is rotated by a suitable DC motor or the like, operating at a speed coordinated with the image capture component. In yet other embodiments, other devices, such as liquid crystals (such as, but not limited to, those manufactured by Meadowlark Optics (Frederick, Co.)) or electro-optic or acousto-optic devices (such as, but not limited to, those manufactured by Hinds Instruments (Hillsboro, Oreg.)) can be used to rotate the polarization of the light. Again, control of the rotation can be done manually by a user or can be controlled by the computer component (which, in turn, is optionally controlled by input from the user).

Detection Devices

In various embodiments herein, reflected light that returns from the tissues/structures being monitored is captured in a detection device. In turn, the detection device typically relays such information to the computer component of the system. The detection device can comprise, e.g., a CCD camera or the like. Those of skill in the art will be familiar with numerous types and examples of detection devices capable of capture of light emission that can optionally be used with embodiments of the current invention. Recitation of a particular type or example of detection device in various illustrations herein should therefore not be taken as limiting. For example, the detecting device of various system/devices herein can comprise, e.g., a PIN photodiode (e.g., operated in either photovoltaic or photocurrent modes), an avalanche photodiode, a phototransistor, a photomultiplier tube, a CCD array, or a CMOS array. Various detector devices herein, depending upon the embodiment, can comprise one or more of elements such as silicon or indium gallium arsenide.

Computer

As noted above, the various components of the systems herein (whether monitoring or treatment systems) can be coupled to an appropriately programmed processor or computer that functions to instruct the operation of these instruments in accordance with preprogrammed or user input instructions, receive data and information from these instruments, and interpret, manipulate and report this information to the user. As such, the computer is typically appropriately coupled to these instruments/components (e.g., including analog to digital or digital to analog converters as needed).

The computer optionally includes appropriate software for receiving user instructions, either in the form of user input into set parameter fields, e.g., in a GUI, or in the form of preprogrammed instructions, e.g., preprogrammed for a variety of different specific operations. The software then converts these instructions to appropriate language for instructing the correct operation to carry out the desired operation (e.g., of light illumination, RF intensity, etc.).

The computer also optionally receives the data from one or more sensors/detectors included within the system, and interprets the data, either provides it in a user understood format (e.g., on a display or computer printout), or uses that data to initiate further instructions, in accordance with the programming, e.g., such as in control of illumination, temperatures, and the like.

In various embodiments of the invention, the computer can include software for the monitoring and control of light illumination and capture, electrical permittivity, heating/cooling of various device components as well as various tissues/areas of a subject (e.g., skin surface, collagen, etc.), etc. Additionally the software can be optionally used to control movement of the illumination/capture footprints over a tissue surface, e.g., in coordination with the treatment being monitored. The computer can also provide instructions, e.g., to any heating/cooling component system, etc.

Any controller or computer optionally includes a monitor which is often a cathode ray tube (“CRT”) display, a flat panel display (e.g., active matrix liquid crystal display, liquid crystal display), or the like. Data produced from the current systems is optionally displayed in electronic form on the monitor. Additionally, the data gathered from the system can be outputted in printed form. The data, whether in printed form or electronic form (e.g., as displayed on a monitor or deposited on tape, CD, or disc), can be in various or multiple formats, e.g., curves, histograms, numeric series, tables, graphs and the like.

Computer circuitry is often placed in a box which includes, e.g., numerous integrated circuit chips, such as a microprocessor, memory, interface circuits. The box also optionally includes a hard disk drive, a floppy disk drive, a high capacity removable drive such as a writeable CD-ROM, and other common peripheral elements. Inputting devices such as a keyboard or mouse optionally provide for input from a user and for user selection of sequences to be compared or otherwise manipulated in the relevant computer system. It will be appreciated that the computer component in the systems/devices herein does not necessarily refer to a Personal Computer (PC), but can also or instead comprise a microcontroller or microprocessor.

Automation

Although the methods of the invention can be performed manually, in particular embodiments, such steps as placement of the components in relation to the subject or cycling RF intensity up or down to reach a desired result are performed in an automated fashion.

Housing

Placement and movement of the various components of the devices/systems herein is optionally controlled and secured by, e.g., an armature, scaffolding, or housing in which the components are located. In particular embodiments, the components, or at least part of the components, are handheld. Handheld and other manipulatable components can be used to move over an area to be monitored (e.g., a subject's skin surface) or within an area to be monitored (e.g., within a subject's body). In some embodiments, one or more component of the system comprises a component that can be arthroscopically or otherwise inserted into a subject. In some embodiments, the systems herein can optionally comprise one or more components to help stabilize and/or locate the one or more other components of the system in relation to the tissue area being monitored. Thus, some embodiments can comprise stabilizers, mounted platforms (e.g., for a subject), straps, etc.

Thus, it will be appreciated that the various components herein (whether the entirety of a system or just part of a system) can be arranged on a scaffolding or framework and optionally enclosed within a housing. The particular configuration of such framework and/or housing can optionally vary in different embodiments based upon, e.g., the particular components, their size, etc. In typical embodiments however, the framework keeps the various components secure and in the proper location and orientation while also optionally aiding in the movement of the components when necessary.

Heating and Cooling

In some embodiments, the systems herein comprise a heating/cooling component (and optionally a heating/cooling control component) having heating/cooling capabilities. In some embodiments, the heating/cooling component can optionally regulate the temperature of the other components of the systems/devices, e.g., the light emitter, the computer, etc. For example, the heating/cooling component can optionally regulate the temperature of the CCD camera. Such temperature control elements can also optionally help regulate the surface of the tissues whose treatment is being monitored. See below.

### Treatment of Tissue by Drastic Temperature Changes

The methods and systems/devices of the invention (both monitoring and treatment systems/devices) can be used in a number of different treatment programs for a number of different medical conditions. Thus, it will be appreciated that recitation of particular treatments or the like herein should not necessarily be taken as limiting. Heat and cold treatments have been widely used in the medical field and numerous well-established therapeutic modalities exist that take advantage of such treatments. See, e.g., Hayashi, et al., “The effect of thermal heating on the length and histologic properties of the glenohumeral joint capsule” Am J Sports Med, 25(1):107-12, 1997; Naseef, et al., “The thermal properties of bovine joint capsule. The basic science of laser- and radiofrequency-induced capsular shrinkage” Am J Sports Med, 25(5):670-4,1997; and Oloff, et al., “Arthroscopic monopolar radiofrequency thermal stabilization for chronic lateral ankle instability: a preliminary report on 10 cases” J Foot Ankle Surg, 39(3):144-53, 2000. Light, ultrasound, radio frequency, shockwaves, magnetism and other forms of energy have been used to modify tissue temperatures in order to achieve benefits such as stimulation of different biological responses, ablation of tissue, or induction of necrosis. For instance, wound healing response stimulation, collagen stimulation, collagen denaturation, collagen contraction, disruption of fat cells for aesthetic purposes, reduction of benign or malign tumors and the like are all medical procedures achieved by temperature variation. The embodiments herein further and/or and utilize such procedures, and others, and increase their usefulness and applicability by allowing monitoring (optionally real time monitoring) of the changes brought about by the treatments and by presenting novel aspects of treatment. Moreover, in many embodiments, the current invention can be non-invasive (or minimally invasive) in regard to the tissue being treated, thus avoiding more damaging monitoring processes such as biopsies.

As indicated throughout, the current invention can, in many embodiments, be used to monitor treatment of collagen structures in various tissues or actually treat collagen in various tissues. Besides water, collagen is the main component of skin, cartilage, and connective tissue (including, ligaments, tendons, and the like). Trauma, ageing, and other clinical entities can damage collagen's structure through thinning and disorientation of collagen fibers, myxoid degeneration, hyaline degeneration, chondroid metaplasia, calcification, vascular proliferation, and fatty infiltration, etc. See, e.g., Hashimoto, et al., “Pathologic evidence of degeneration as a primary cause of rotator cuff tear” Clin Orthop Relat Res, (415):111-20, 2003.

As is well known in the art, thermotherapy can cause collagen denaturation and contraction by well-known mechanisms of action. See, e.g., Allain, et al., “Isometric tension developed during heating of collagenous tissues. Relationships with collagen cross-linking” Biochim Biophys Acta, 533(1):147-55, 1978; Allain, et al., “Isometric tensions developed during the hydrothermal swelling of rat skin” Connect Tissue Res, 7(3):127-33, 1980; Chen, et al., “Heat-induced changes in the mechanics of a collagenous tissue: isothermal, isotonic shrinkage” J Biomech Eng, 120(3):382-8, 1998; Cooper, et al., “Correlation of thermal properties of some human tissue with water content” Aerosp Med, 42(1):24-7, 1971; Hayashi, et al., “The biologic response to laser thermal modification in an in vivo sheep model” Clin Orthop Relat Res, (373):265-76, 2000; Hayashi, et al., “The effect of nonablative laser energy on joint capsular properties. An in vitro mechanical study using a rabbit model” Am J Sports Med, 23(4):482-7, 1995; Hayashi, et al., “The mechanism of joint capsule thermal modification in an in-vitro sheep model” Clin Orthop Relat Res, (370):236-49, 2000; and Hayashi, et al., “The effect of nonablative laser energy on the ultrastructure of joint capsular collagen” Arthroscopy, 12(4):474-81, 1996.

The use of collagen denaturation/contraction through energy-based treatment modalities can be used in a number of important applications in fields ranging from neurosurgery and orthopedics to ophthalmology, aesthetic medicine, and surgery of the gastrointestinal tract. See, e.g., Avitall, et al., “Physics and engineering of transcatheter cardiac tissue ablation” J Am Coll Cardiol, 22(3):921-32, 1993; Avitall, et al., “The safety and efficacy of multiple consecutive cryo lesions in canine pulmonary veins-left atrial junction” Heart Rhythm, 1(2):203-9, 2004; Avitall, et al., “Linear lesions provide protection from atrial fibrillation induction with rapid atrial pacing” J Cardiovasc Electrophysiol, 13(5):455-62, 2002; Brodkey, et al., “Reversible Heat Lesions With Radiofrequency Current. A Method Of Stereotactic Localization” J Neurosurg, 21:49-53, 1964; Daoud, et al., “Catheter ablation of ventricular tachycardia” Curr Opin Cardiol, 10(1):21-5, 1995; Lesh, “Interventional electrophysiology--state-of-the-art” Am Heart J, 126(3 Pt 1):686-98, 1993; Moraci, et al., “Trigeminal neuralgia treated by percutaneous thermocoagulation. Comparative analysis of percutaneous thermocoagulation and other surgical procedures” Neurochirurgia (Stuttg), 35(2):48-53, 1992; Saxon, et al., “Results of radiofrequency catheter ablation for atrial flutter” Am J Cardiol, 77(11):1014-6, 1996; Seegenschmiedt, et al., “The current role of interstitial thermo-radiotherapy” Strahlenther Onkol, 168(3):119-40, 1992; and Sweet, et al., “Controlled thermocoagulation of trigeminal ganglion and rootlets for differential destruction of pain fibers. 1. Trigeminal neuralgia” J Neurosurg, 40(2):143-56, 1974. Those of skill in the art will be familiar with other applications/procedures utilizing temperature induced denaturation/contraction of collagen. Such applications/procedures are optionally monitored through use of particular methods and systems of the current invention, while other methods and systems of the invention can achieve such applications/procedures through the treatment aspects herein.

One common procedure to denature/contract collagen uses temperature generated through radiofrequency energy. Such energy can be applied to an area to be treated, e.g., through direct contact with the targeted structure via a surgical incision. Recently, some technologies have offered the ability of providing therapeutic heat levels through an overlying tissue (e.g., heat/energy applied transcutaneously, transsynovially, transmucosally, transintimaly, etc.) with the goal not only of treating the desired collagen but also preserving the integrity of non-targeted structures (e.g., skin, subcutaneous tissue, and other tissues depending on the targeted structure). See, e.g., Chen, et al., “Heat-induced changes in the mechanics of a collagenous tissue: isothermal, isotonic shrinkage. J Biomech Eng,” 120(3):382-8, 1998; Naseef, et al., “The thermal properties of bovine joint capsule. The basic science of laser- and radiofrequency-induced capsular shrinkage” Am J Sports Med,” 25(5):670-4, 1997; and Oloff, et al., “Arthroscopic monopolar radiofrequency thermal stabilization for chronic lateral ankle instability: a preliminary report on 10 cases” J Foot Ankle Surg, 39(3):144-53, 2000. However, visualization of the treated structure has previously been difficult and, even when direct visualization is possible, the fact that most collagen changes are not visible to the naked eye is problematic. Various embodiments of the current invention are especially useful in monitoring such procedures. See below. The invention recognizes the baseline condition of a structure while also determining the impact of the provided treatment on the structure and optionally on other underlying or overlying structures. For example, by distinguishing between the collagen in superficial layers of tissue and the collagen in deeper structures, some embodiments of the current invention can aid in defining the clinical endpoint of a treatment based on changes to the structure being treated and/or on changes to nearby structures other embodiments herein present novel methods and systems/devices involved in RF treatment of collagen. Other embodiments herein aid in control of various RF treatments through improved electrode design (temperature controlled, disposable tipped electrodes, contact sensing aspects, etc.).

The fundamental unit of collagen consists of tropocollagen polypeptides organized into a triple helix. This triple helical structure is stabilized by intramolecular bonds, principally hydrogen bonds. The triple helices are further organized by intermolecular bonding. See, e.g., Arnoczky, et al., “Thermal modification of connective tissues: basic science considerations and clinical implications” J Am Acad Orthop Surg, 8(5):305-13, 2000. In tissues such as ligaments and tendons, the association of neighboring helices is largely parallel, resulting in unidirectional strands, while in other tissues such as capsular and dermal collagen, the collagen helices are less unidirectional, being instead confined within a plane.

Application of heat to tissue collagen in the range of 60 to 90° C., results in denaturation of the higher order protein structure. The intramolecular hydrogen bonds stabilizing the triple helices are particularly susceptible to disruption, while the intermolecular bonds are generally more heat stable. Therefore, application of heat has the effect of unraveling the triple helical structure of collagen while maintaining overall strand integrity. The result for ligaments, tendons, and other linearly oriented collagen structures is shortening of the collagen along the long axis and thickening of the collagen along the short axis. See, Arnoczky, supra. For collagen oriented within a plane, such as capsular and dermal collagen, contraction of the planar sheet is observed in response to heat, with thickening in the direction perpendicular to the plane.

During thermal treatments of tissue, the extent of collagen denaturation/contraction is affected by the temperature reached and/or the duration of the treatment provided. See Wall, et al., “Thermal modification of collagen.” J Shoulder Elbow Surg, 8(4):339-44, 1999. In addition, due to the variation in collagen composition, particularly the extent of intramolecular cross-linking, the extent of collagen denaturation/contraction can be difficult to predict in advance even if the temperature profile is fully characterized. Thus, various embodiments of the current invention are beneficial to such treatments because they can monitor the presence and extent of any denaturation that occurs during or as a result of treatment and thereby allows medical practitioners to more accurately administer treatment.

Monitoring of Tissued Changes Arising from Chemical/Cosmeceutical Use

Treatment of tissues through epidermal/dermal or percutaneous application of lotions, creams, or other substances is wide spread in the general populace and within the medical community. Many conditions can be treated by the application of different chemicals that are anticipated to induce changes in tissues including the skin, subcutaneous fat, or connective structures. See, e.g., Fang, et al., “Efficacy and irritancy of enhancers on the in-vitro and in-vivo percutaneous absorption of curcumin” J Pharm Pharmacol, 55(5):593-601, 2003, and Schottelius, et al., “An aspirin-triggered lipoxin A4 stable analog displays a unique topical anti-inflammatory profile” J Immunol, 169(12):7063-70, 2002. For instance, the cosmetics industry provides a wide variety of so called “cosmeceuticals” to increase collagen production. See, e.g., Katayama, et al., “A pentapeptide from type I procollagen promotes extracellular matrix production” J. Biol. Chem., 268(14):9941-9944, 1993. However, no objective measurement of effectiveness has previously been available to users. Thus, various embodiments of the current invention can optionally be used to monitor changes, if any, caused by use of cosmeceuticals or the like. For example, using some embodiments of the present invention, a baseline measurement can be taken of the collagen status of a tissue, the cosmeceutical or other putative treatment can then be applied/performed and additional measurements of the collagen status can be performed to detect any change. Again, it will be appreciated that the invention can monitor change before the treatment, during the treatment, and/or after the treatment of the tissue.

### Use of Embodiments of the Invention with Various Treatments

As will readily be appreciated by those of skill in the art, embodiments of the current invention can be used as a diagnostic and/or research tool or in conjunction with therapeutic/prophylactic modalities that aim at changing and/or monitoring collagen characteristics. In such embodiments the invention can measure collagen's status in various ways at a baseline and can be used for repeated measurements to establish changes in collagen content or characteristics within the studied tissue to determine the impact of a given intervention. As stated above, the treatment that is tracked can treat one or more layers of tissue (e.g., collagen). In situations where only one structural layer is treated, such layer can be superficial to a structural layer that is to not be treated or vice versa. The treatments monitored can be those that induce wound healing, induce collagen denaturation/renewal, induce collagen deposition, etc. Again, recitation of particular treatment methods/goals should not necessarily be taken as limiting. Additionally, the embodiments herein concerning wound healing, electrode design, etc. can also be used in conjunction with numerous treatments for diagnosis, research, treatment, etc.

### Kits and Articles of Manufacture

In some embodiments, the invention provides a kit or an article of manufacture containing materials useful for the methods described herein and/or comprising examples of the systems/devices described herein. Such kits can optionally comprise one or more containers, labels, and instructions, as well components for monitoring of treatment.

The kits can also optionally comprise one or more light sources, polarizers, lenses, polarization rotators, fiber optics, light detectors, RF generators, computers, etc. as well as optionally other components. The kits can optionally include scaffolding, armature or other organizational structures to controllably position and/or move the various components of the systems/devices of the invention.

In many embodiments, the kits comprise instructions (e.g., typically written instructions) relating to the use of the kit to determine and/or monitor changes in tissue (e.g., collagen). In some embodiments, the kits comprise a URL address or phone number or the like for users to contact for instructions or further instructions.

Treatment and Monitoring within Cavities

The present embodiment is related to collagen content and collagen denaturation sensors and/or systems, more specifically a collagen content and collagen denaturation process sensor and/or system that can be used within natural cavities (e.g., knee joint, GI tract, etc.) or created cavities (e.g., around tendons and ligaments, tumors, etc.) within a subject. The embodiment also includes methods of use of such sensors/systems, e.g., in conjunction with endoscopic diagnostic or therapeutic interventions.

It has historically been difficult to establish real time collagen content and monitor collagen changes in tissues within cavities (real, virtual, or created). This lack of knowledge has posed a serious limitation to the clinician to determine if a given intervention aimed at changing collagen is indicated and to determine the extension of the intervention on such tissue.

Therefore in various embodiments, the invention provides a sensor system for detecting collagen content and collagen variations (i.e., collagen status) within cavities including at least a signal generator, a connector, and a monitor and/or computer processor. The signal generator generates a signal (e.g., optic, electrical, etc., see throughout), which may be carried by the connector to the tissue to be analyzed and then back to the monitor and/or processor, which generates an output corresponding to the signal received and which indicates the content and/or status of the collagen. The invention also provides methods of measuring collagen content and collagen variations in tissue by inserting at least a portion of some part of the sensor system (e.g., all or part of the connector (which in some embodiments comprises a probe/shaft) or all or part of the signal generator, etc.) into a cavity of a subject, such as a human, until it reaches the collagen containing structure to be analyzed, then measuring collagen content using the sensor.

It will be appreciated that the various monitoring embodiments herein can optionally be used in conjunction with any of the various treatment embodiments herein, e.g., as concurrent and/or complementary applications. In some instances, the monitoring and treatment embodiments (e.g., any combination of embodiments herein) can be integrated into a single device or system, while in other instances the monitoring and treatment embodiments can be in separate devices or systems but used together (again, e.g., concurrently, sequentially, or in a complementary fashion, etc.). Again, the embodiments herein can also be used with various embodiments found within U.S. Ser. No. 61/066,593 filed Feb. 20, 2008; PCT/US2009/001093 filed Feb. 20, 2009; U.S. Ser. No. 12/380,014 filed Feb. 20, 2009; U.S. Ser. No. 61/274,704 filed Aug. 19, 2009; U.S. Ser. No. 12/806,811 filed Aug. 19, 2009; U.S. Ser. No. 61/339,786 filed Mar. 8, 2010.

Thus, the present embodiments comprise a sensor system to monitor collagen content and/or collagen alterations within cavities within a subject. The sensor system can optionally generate a signal corresponding to collagen content when placed adjacent to a collagen containing tissue (e.g., within a cavity of a subject). The sensor system can also include a connector operably connected to the signal generator to carry the signal (e.g., optical, electrical, etc.) from the generator to the tissue and also to carry the return signal (e.g., optical, electrical) back from the tissue to a monitor, computer processor, or data storage unit, etc., and a monitor and/or computer processor operably connected to the connector to receive the signal and generate an output corresponding to the signal.

In some embodiments, the sensor system can include components for sensing collagen content when placed adjacent to a collagen containing tissue (again, e.g., within a cavity within a subject) and which components are capable of generating a signal corresponding to collagen content or status in the tissue. The sensor system can also include various components for carrying the signal (e.g., to a display, etc.) and components for receiving the signal and generating an output corresponding to the signal.

In some embodiments, a sensor system can be provided which can include a signal generator operable to sense collagen content and generate a signal corresponding to collagen content when placed adjacent to collagen contenting tissue. The sensor system can also include at least a segment of a connector coupled to the signal generator and operable to carry the signal.

In some embodiments, the invention can include multiple signal generators, connectors, etc. The sensor system can include signal generation components that are operable to sense collagen content and generate a signal corresponding to collagen content when placed adjacent to a bronchial tissue. The sensor can include at least a portion of a connector component coupled to the signal generator and operable to carry the signal.

The invention also includes embodiments comprising methods of measuring collagen content in a given tissue (e.g., within a cavity of a subject) and/or determining collagen status in a given tissue (again, e.g., within a cavity of a subject). The methods can include inserting part of the sensor system (e.g., all or part of the signal generator and/or all or part of the connector) into a cavity of the subject, lodging or placing such components in the cavity such that at least a portion of the components is adjacent to a collagen containing tissue, generating a signal (e.g., optical, electrical, etc.) and transmitting it to the tissue, collecting the return signal from the tissue and conveying it to the monitor and/or computer process or microchip processor so that an output can be provided on/through the monitor that provides information corresponding to collagen content or status. In some of such embodiments, the methods can include connecting a replaceable sensor component to the sensor system prior to use. In other words, in some embodiments of the methods and devices, the signal generator and/or the connector (or at least part of it) can be replaceable. Thus, the replaceable part can optionally be changed between uses (e.g., between subjects), can be changed for different uses (e.g., when changing monitoring or treatment regimens), etc. The replaceable part is also optionally reusable. For example, some such parts can be removed, sterilized, and reused. Methods for tracking the extent of use or age of the replaceable parts, such as by use of an embedded electronic chip, are also optionally included in some embodiments. The replacement part can comprise a probe or shaft component of the connector component. See below.

In some embodiments, the methods can include use of a component that is hollow (or at least part of which is hollow) so to allow fluids to flow distal from its anchoring point. In some embodiments the methods can include components that can be incorporated into a variety of surgical and endoscopic and endoscopic-surgery tools, electrodes such as radiofrequency electrodes, or any other tools used to either to diagnose or treat different conditions within a given cavity (e g., tumors, and tumor like conditions, traumatic, inflammatory and the like. Thus, the embodiments of this aspect can optionally be integrated within or with other surgical tools, etc.

In some embodiments the component(s) that came into contact with the subject can be removed from the subject after providing information and then those components can be disconnected from the rest of the sensor system.

The tracking embodiments of the invention can help ensure that a desired treatment outcome has been reached and that the risks associated with unnecessary heating are avoided during treatment. In addition, the ability to determine baseline collagen “content” in tissue may become a predictor of success or even better, a prequalification for a given therapeutic intervention aimed at collagen molecules in such a way that “good candidates” and “bad candidates” may be identified and physicians and patients may become aware of the real potential of the treatment. Another potential application may be that of establishing the truth of claims of the so-called “cosmeceuticals” and other therapeutic modalities regarding their ability to improve collagen content in tissue.

Following the establishment of a sufficient database and knowing some variables such as race, gender and age, the technology of the embodiments of the invention, both this and other embodiments herein, can become diagnostic tools in which a given “number” or “grade” may allow physicians to better understand and guide treatments or even provide an overall health parameter of nutrition and condition. In fact, a direct correlation of collagen content in the skin with that of bones has been established. Because of the comparable changes in skin and bones, it may be possible to use skin collagen as a predictor of the state of bones and their response to treatment in a quick and inexpensive manner. Again, it should be appreciated that the above discussed concerns and points are optionally applicable in review or consideration of other embodiments herein and not just embodiments discussed in this section. The present embodiment aids in such treatment by better allowing monitoring of collagen status/changes within cavities of a subject.

Many therapeutic procedures such as thermal capsulorrhaphy, skin tightening, and others ideally should be controllable and predictable. Appropriate patient selection and real-time monitoring of tissular changes to avoid under or over-treatment may result in a higher level of success and less complications. The transformational value of the current embodiments represents a breakthrough for all energy-based technologies in general and radiofrequency generators in particular.

Either integrated within current electrodes (invasive, minimally invasive, or non-invasive) or other treatment devices, or as a standalone tool, the current embodiments can increase the usefulness and applicability of energy-based therapeutic interventions by monitoring the changes brought about by the treatments.

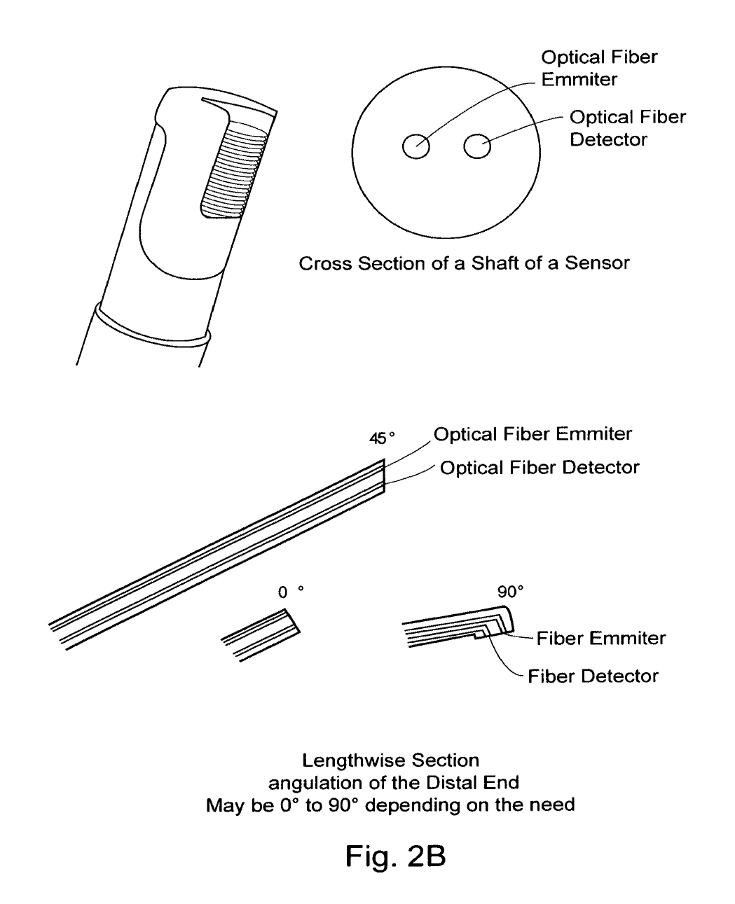

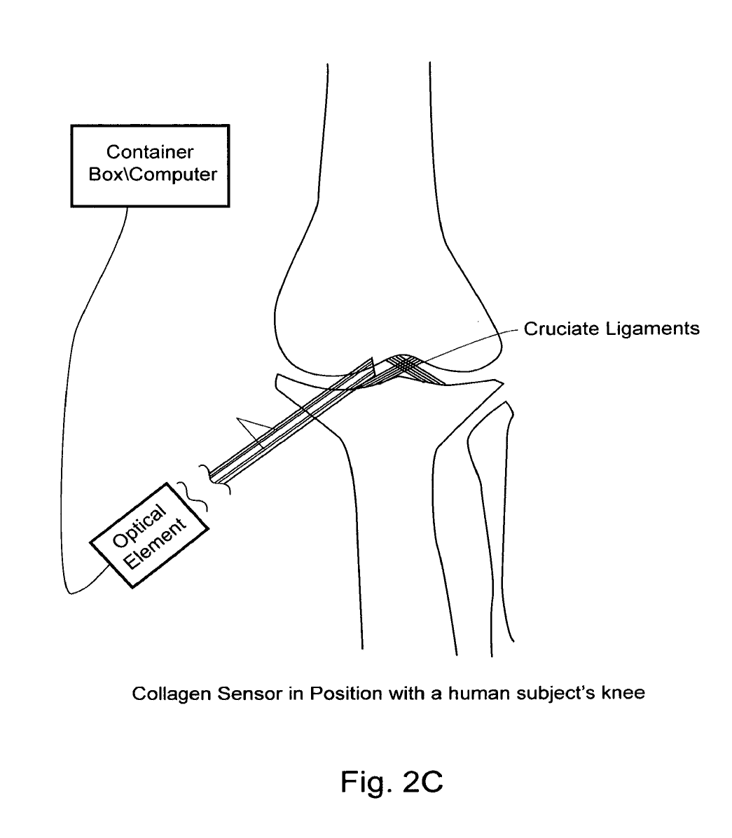

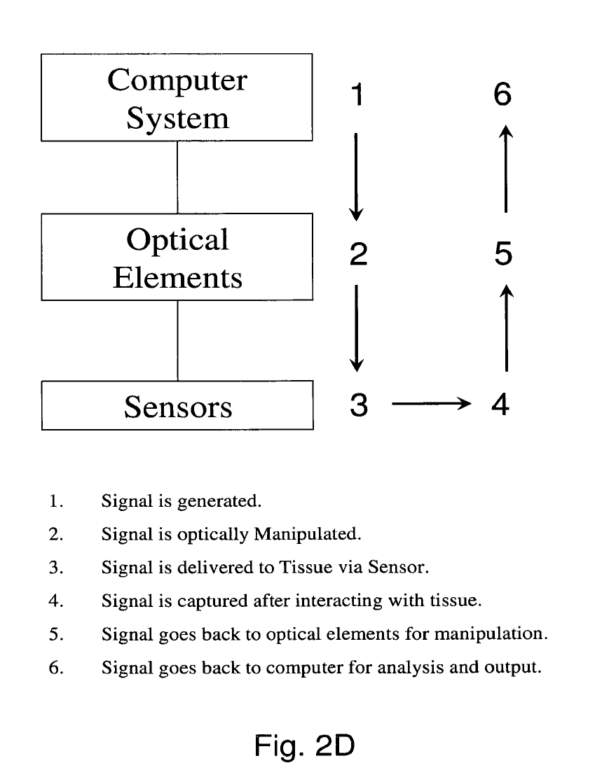

As can be seen, FIG. 2a presents a diagram of a collagen containing area, a knee joint, that comprises collagen containing structures which can be measured and monitored with the current embodiments.

FIG. 2b shows a diagram of a cross section of a shaft of an exemplary collagen sensor system as well as a lengthwise view of the same. As can be seen, some embodiments of the systems can comprise a shaft (or probe, etc.) and can include an optical fiber emitter and an optical fiber detector, e.g., for embodiments which use optical methods to monitor collagen (see above). Other embodiments can, of course, comprise other monitoring methods such as electrical permittivity. In such embodiments, the sensor can comprise an electrical emitter and detector (e.g., to measure permittivity). It can also be seen that the angle of the distal end of the shaft/probe can be different degrees in different embodiments, e.g., flat (0), beveled (e.g., 45), or even 90 degrees. In some such embodiments, the shaft/probe can comprise mirrors or prisms to facilitate angulation. The figure also presents a picture of an exemplary optical detector embodiment of the invention (e.g., utilizing polarization preserving fibers, etc.).

FIG. 2c is a simplified diagram of a collagen sensor system in a sensing position in a human subject (monitoring collagen within a knee). It will be appreciated that the sensor system can include a probe/shaft, etc. for use in cavities within a subject. Furthermore, the signal generator (whether, e.g., optical or electrical, etc.) can optionally be comprised within the probe/shaft component inserted within a subject or can be separate but operably connected to the inserted components.

FIG. 2d is a block diagram of a simplified process for using a collagen sensor system according to the current embodiments. Again, the sensor system here would can include a probe/shaft for monitoring within a cavity of a subject. Other embodiments can include “free-space” embodiments where optical signals are transmitted and received not through optical fibers or the like.

In various embodiments, the invention comprises a sensor system which comprises a signal generator that can generate a signal that can be transmitted to a tissue and which return signal from the tissue can be collected to create an output (e.g., reading, number, graph, etc.) corresponding to collagen content or status in the tissue and its variation due to therapeutic interventions or changes over time. The signal generator comprises and/or is operably connected to a connector component, optionally having a probe/shaft capable of insertion into a cavity within a subject. The embodiments also include a connector operable to carry energy to the area to be monitored (e.g., light, etc. in embodiments utilizing optical monitoring or electricity in embodiments using permittivity) and signals returning from the monitored area (e.g., reflected light, etc. from the collagen structure in embodiments utilizing optical monitoring or electricity in embodiments using permittivity) and a monitor and/or computer or microprocessor that can receive the signal and generate an output corresponding to the signal that can be interpreted/read by a user.

In the various embodiments, the collagen tissue that is monitored can be, e.g., a tendon, a ligament or a capsule. Also in the various embodiments, the signal that is generated/measured/monitored can be one or more of, e.g., an optical signal, near infrared light, an analog signal, a digital signal, or an electrical signal.

In the various embodiments, the sensor system can comprise one or more of, e.g., a collagen detecting probe, an indicator chemical (e.g., one which changes, e.g., color based on changes in its milieu), an optical fiber, an electrically conductive material, a microchip, or a signal generator. In many embodiments, the at least a portion of the sensor system can be inserted into a cavity of a subject, typically, but not necessarily exclusively via a probe/shaft component.

In various embodiments, the connector of the system can comprise one or more of, e.g., an optical fiber or an electrically conductive material. In various embodiments, the connector and its optional shaft/probe have pathways for light/energy/electricity to travel to the area to be monitored as well as pathways for light/energy/electricity to travel back from the area monitored to the rest of the sensor system so to be measured/interpreted. The connector can further comprise a shaft or probe connected to the sensor system. In such embodiments, the shaft optionally can be inserted into a body cavity of a subject to, or near by to, a collagen-containing structure of the subject.

In some embodiments the connector can further comprise a cable connected to the shaft/probe, the signal generator, the monitor, and/or the computer or microprocessor.

Some embodiments herein comprise systems comprising one or more of: wherein the connector comprises at least one coupler (e.g., an adaptor or connecter); wherein the monitor comprises a display; wherein the monitor comprises an alarm operable to be triggered based on the output indicating a collagen level or status; and wherein the monitor comprises a microchip. In some embodiments, various components, e.g., those components or portions thereof that come into contact with a subject, are replaceable and/or reusable. For example, sensor segments (e.g., probes) inserted within a subject can be wholly or partially replaceable to ensure proper sterility, etc. In some embodiments, the replaceable sections/components can be resterilized and reused.

Various embodiments herein can also further comprise at least one component operable to convert an optical signal to a digital signal, an optical signal to an analog signal, a digital signal to an analog signal, or vice versa. Also, some embodiments can further comprise at least one replaceable component, e.g., a sensor, one or more segment of a sensor, a connector, one or more segment of a connector, etc.

In another aspect, the invention comprises embodiments having a sensor system comprising components for sensing collagen content in tissue when placed adjacent to a collagen containing tissue; components for generating a signal corresponding to collagen content; components for carrying the signal; and components for receiving the signal and generating an output corresponding to the signal. Such systems can optionally be used to sense collagen in tissues such as tendons, capsules, or ligaments. In such embodiments, the signal can optionally comprise one or more of, e.g., an optical signal, a near infrared light, an analog signal, a digital signal or an electrical signal.

In yet other aspects, the invention comprises sensor systems that are operable to sense collagen content or status by generating an output corresponding to collagen content when at least part of the system is placed adjacent to a collagen containing tissue. The systems can optionally sense collagen content in tissues such as tendons, ligaments, and capsules. In such embodiments, the signal can optionally comprise one or more of, e.g., an optical signal, an analog signal, a digital signal, an electrical signal, or any combinations thereof. Furthermore, the signals can comprise one or more of, e.g., near infrared light, an optical signal, or an electrical signal, and the signal generator can comprise a microchip. The embodiments in such aspects can also further comprise an outer portion of one or more component that acts to protect the component, e.g., the signal generator, and can also optionally comprise an indicator. The various components that transmit signals (e.g., a probe to send and receive signals to and from a collagen structure) can comprise an ellipsoid cross-sectional shape and can optionally comprise a diameter of from about 1 mm to about 6 mm. In some instances, the sensor system can further comprise at least a segment of a connector coupled to the signal generator that can carry the signal. The portions of the system that are optionally reusable can optionally be repaired, resterilized, etc., before reuse.

In other aspects, the invention includes methods of measuring collagen content and status and collagen variations in a subject by inserting a component or a portion thereof (e.g., a probe/shaft) and at least a segment of a connector into a cavity of the subject; lodging the component into said cavity such that at least a portion of the component is adjacent to a collagen-containing tissue; generating a signal and transmitting it to the collagen containing tissue and receiving a return signal from the tissue; conveying the return signal from the via the connector; conveying the signal from the connector to a monitor and/or a computer processor; and providing information corresponding to collagen content or status or collagen variation in the tissue using the signal and the monitor/processor. Such methods can be used on human subjects. In such embodiments, providing information can include displaying numeric data on a display of the monitor and/or triggering an alarm when collagen changes have reached a given target. The methods can further comprise connecting a replaceable component to a sensor system prior to use of the system and/or removing the replaceable component from the subject after providing information; and disconnecting the replaceable component from the sensor system.

Various references useful for the embodiments herein, as well as optionally for other sections and embodiments herein, include, e.g., References: AAOS. Use of Thermal Modalities (Lasers and Radiofrequency Devices) in Orthopaedic Surgery. Available via internet at URL “www6.aaos.org/news/PDFopen/PDFopen.cfm?page_url=http://www.aaos.org/about/pape rs/advistmt/1010.asp”. Accessed Sep. 21, 2009, 2009; Abraham M T, Chiang S K, Keller G S, Rawnsley J D, Blackwell K E, Elashoff D A. Clinical evaluation of non-ablative radiofrequency facial rejuvenation. J Cosmet Laser Ther. 2004; 6(3):136-144; Abrams J S. Thermal capsulorrhaphy for instability of the shoulder: concerns and applications of the heat probe. Instructional course lectures. 2001; 50:29-36; Allain J C, Le Lous M, Cohen S, Bazin S, Maroteaux P. Isometric tensions developed during the hydrothermal swelling of rat skin. Connect Tissue Res. 1980; 7(3):127-133; Alster T S, Tanzi E. Improvement of neck and cheek laxity with a nonablative radiofrequency device: a lifting experience. Dermatol Surg. 2004; 30(4 Pt 1):503-507; discussion 507; Arnoczky S P, Aksan A. Thermal modification of connective tissues: basic science considerations and clinical implications. The Journal of the American Academy of Orthopaedic Surgeons. 2000; 8(5):305-313; BlueShield B. BlueCross BlueShield Association Medical Policy Reference Manual, Policy 7.01.82; Burns J. Thermage: Monopolar radiofrequency. 2005; 25(6):638-642; Caprise P A, Jr., Sekiya J K. Open and arthroscopic treatment of multidirectional instability of the shoulder. Arthroscopy. 2006; 22(10):1126-1131; Carter T R. Anterior cruciate ligament thermal shrinkage. Clinics in sports medicine. 2002; 21(4):693-700, ix; Carter T R, Bailie D S, Edinger S. Radiofrequency electrothermal shrinkage of the anterior cruciate ligament. The American journal of sports medicine. 2002; 30(2):221-226; Chang J H, Hsu A T, Lee S J, Chang G L. Immediate effect of thermal capsulorrhaphy on glenohumeral joint mobility. Clin Biomech (Bristol, Avon). 2004; 19(6):572-578; Chen S, Haen P S, Walton J, Murrell G A. The effects of thermal capsular shrinkage on the outcomes of arthroscopic stabilization for primary anterior shoulder instability. The American journal of sports medicine. 2005; 33(5):705-711; Chen S S, Wright N T, Humphrey J D. Heat-induced changes in the mechanics of a collagenous tissue: isothermal, isotonic shrinkage. Journal of biomechanical engineering. 1998; 120(3):382-388; Chvapil M J L. The shrinkage temperature of collagen fibres isolated from the tail tendons of rats of various ages and from different places of the same tendon. Gerontologia 1963. 1963; 1:18-29; D'Alessandro D F, Bradley J P, Fleischli J E, Connor P M. Prospective evaluation of thermal capsulorrhaphy for shoulder instability: indications and results, two- to five-year follow-up. The American journal of sports medicine. 2004; 32(1):21-33; Dodds S D, Panjabi M M, Daigneault J P. Radiofrequency probe treatment for subfailure ligament injury: a biomechanical study of rabbit ACL. Clin Biomech (Bristol, Avon). 2004; 19(2):175-183; Farng E, Hunt S A, Rose D J, Sherman O H. Anterior cruciate ligament radiofrequency thermal shrinkage: a short-term follow-up. Arthroscopy. 2005; 21(9):1027-1033; Gerber A, Warner J J. Thermal capsulorrhaphy to treat shoulder instability. Clinical orthopaedics and related research. 2002 (400):105-116; Gieringer R E. Arthroscopic monopolar radiofrequency thermal capsulorrhaphy for the treatment of shoulder instability: a prospective outcome study with mean 2-year follow-up. Alaska Med. 2003; 45(1):3-8; Greis P E, Burks R T, Schickendantz M S, Sandmeier R. Axillary nerve injury after thermal capsular shrinkage of the shoulder. Journal of shoulder and elbow surgery/American Shoulder and Elbow Surgeons . . . [et al. 2001; 10(3):231-235; Gryler E C, Greis P E, Burks R T, West J. Axillary nerve temperatures during radiofrequency capsulorrhaphy of the shoulder. Arthroscopy. 2001; 17(6):567-572; Hayashi K, Markel M D. Thermal capsulorrhaphy treatment of shoulder instability: basic science. Clinical orthopaedics and related research. 2001 (390):59-72; Hayashi K, Nieckarz J A, Thabit G, 3rd, Bogdanske J J, Cooley A J, Markel M D. Effect of nonablative laser energy on the joint capsule: an in vivo rabbit study using a holmium:YAG laser. Lasers in surgery and medicine. 1997; 20(2):164-171; Hovis W D, Dean M T, Mallon W J, Hawkins R J. Posterior instability of the shoulder with secondary impingement in elite golfers. The American journal of sports medicine. 2002; 30(6):886-890; Indelli P F, Dillingham M F, Fanton G S, Schurman D J. Monopolar thermal treatment of symptomatic anterior cruciate ligament instability. Clinical orthopaedics and related research. 2003 (407):139-147; Khan A S, Sherman O H, DeLay B. Thermal treatment of anterior cruciate ligament injury and laxity with its imaging characteristics. Clinics in sports medicine. 2002; 21(4):701-711, ix; Le Lous M C-S L, Allain J C, Bonaventure J, Maroteaux P. Age related evolution of stable collagen reticulation in human skin. Connect Tissue Res. 1985; 13(145-155); Lephart S M, Myers J B, Bradley J P, Fu F H. Shoulder proprioception and function following thermal capsulorraphy. Arthroscopy. 2002; 18(7):770-778; Levine W N, Bigliani L U, Ahmad C S. Thermal capsulorrhaphy. Orthopedics. 2004; 27(8):823-826; Levitz C L, Dugas J, Andrews J R. The use of arthroscopic thermal capsulorrhaphy to treat internal impingement in baseball players. Arthroscopy. 2001; 17(6):573-577; Levy O, Wilson M, Williams H, Bruguera J A, Dodenhoff R, Sforza G, Copeland S. Thermal capsular shrinkage for shoulder instability. Mid-term longitudinal outcome study. J Bone Joint Surg Br. 2001; 83(5):640-645; Lopez M J, Hayashi K, Fanton G S, Thabit G, 3rd, Markel M D. The effect of radiofrequency energy on the ultrastructure of joint capsular collagen. Arthroscopy. 1998; 14(5):495-501; Lopez M J, Hayashi K, Vanderby R, Jr., Thabit G, 3rd, Fanton G S, Markel M D. Effects of monopolar radiofrequency energy on ovine joint capsular mechanical properties. Clinical orthopaedics and related research. 2000 (374):286-297; Lu Y, Bogdanske J, Lopez M, Cole B J, Markel M D. Effect of simulated shoulder thermal capsulorrhaphy using radiofrequency energy on glenohumeral fluid temperature. Arthroscopy. 2005; 21(5):592-596; McCarty E C, Warren R F, Deng X H, Craig E V, Potter H. Temperature along the axillary nerve during radiofrequency-induced thermal capsular shrinkage. The American journal of sports medicine. 2004; 32(4):909-914; McKeon B, Baltz M S, Curtis A, Scheller A. Fluid temperatures during radiofrequency use in shoulder arthroscopy: A cadaveric study. Journal of shoulder and elbow surgery/American Shoulder and Elbow Surgeons . . . [et al. 2006; Miniaci A, Codsi M J. Thermal capsulorrhaphy for the treatment of shoulder instability. The American journal of sports medicine. 2006; 34(8):1356-1363; Naseef G S, 3rd, Foster T E, Trauner K, Solhpour S, Anderson R R, Zarins B. The thermal properties of bovine joint capsule. The basic science of laser- and radiofrequency-induced capsular shrinkage. The American journal of sports medicine. 1997; 25(5):670-674; O'Neill T, Innes J F. Treatment of shoulder instability caused by medial glenohumeral ligament rupture with thermal capsulorrhaphy. J Small Anim Pract. 2004; 45(10):521-524; Oloff L M, Bocko A P, Fanton G. Arthroscopic monopolar radiofrequency thermal stabilization for chronic lateral ankle instability: a preliminary report on 10 cases. J Foot Ankle Surg. 2000; 39(3):144-153; Philippon M J. The role of arthroscopic thermal capsulorrhaphy in the hip. Clinics in sports medicine. 2001; 20(4):817-829; Savoie F H, 3rd, Field L D. Thermal versus suture treatment of symptomatic capsular laxity. Clinics in sports medicine. 2000; 19(1):63-75, vi; Sekiya J K, Ong B C, Bradley J P. Thermal capsulorrhaphy for shoulder instability. Instructional course lectures. 2003; 52:65-80; Selecky M T, Tibone J E, Yang B Y, McMahon P J, Lee T Q. Glenohumeral joint translation after arthroscopic thermal capsuloplasty of the posterior capsule. Journal of shoulder and elbow surgery/American Shoulder and Elbow Surgeons . . . [et al. 2003; 12(3):242-246; Selecky M T, Tibone J E, Yang B Y, McMahon P J, Lee T Q. Glenohumeral joint translation after arthroscopic thermal capsuloplasty of the rotator interval. Journal of shoulder and elbow surgery/American Shoulder and Elbow Surgeons . . . [et al. 2003; 12(2):139-143; Shuster S. Osteoporosis, a unitary hypothesis of collagen loss in skin and bone. Medical hypotheses. 2005; 65(3):426-432; Sukal S A, Geronemus R G. Thermage: the nonablative radiofrequency for rejuvenation. Clin Dermatol. 2008; 26(6):602-607; Takahashi N, Tasto J P, Ritter M, Ochiai N, Ohtori S, Moriya H, Amiel D. Pain relief through an antinociceptive effect after radiofrequency application. The American journal of sports medicine. 2007; 35(5):805-810; Tasto J P. The role of radiofrequency-based devices in shaping the future of orthopedic surgery. Orthopedics. 2006; 29(10):874-875; Tasto J P, Cummings J, Medlock V, Hardesty R, Amiel D. Microtenotomy Using a Radiofrequency Probe to Treat Lateral Epicondylitis Arthroscopy: The Journal of Arthroscopic & Related Surgery. 2005; 21(7):851-860; Taverna E, Battistella F, Sansone V, Perfetti C, Tasto J P. Radiofrequency-based plasma microtenotomy compared with arthroscopic subacromial decompression yields equivalent outcomes for rotator cuff tendinosis. Arthroscopy. 2007; 23(10):1042-1051; Tibone J E, Lee T Q, Black A D, Sandusky M D, McMahon P J. Glenohumeral translation after arthroscopic thermal capsuloplasty with a radiofrequency probe. Journal of shoulder and elbow surgery/American Shoulder and Elbow Surgeons . . . [et al. 2000; 9(6):514-518; Vangsness C T, Jr., Mitchell W, 3rd, Nimni M, Erlich M, Saadat V, Schmotzer H. Collagen shortening. An experimental approach with heat. Clinical orthopaedics and related research. 1997 (337):267-271; Victoroff B N, Deutsch A, Protomastro P, Barber J E, Davy D T. The effect of radiofrequency thermal capsulorrhaphy on glenohumeral translation, rotation, and volume. Journal of shoulder and elbow surgery/American Shoulder and Elbow Surgeons . . . [et al. 2004; 13(2):138-145; Wall M S, Deng X I I, Torzilli P A, Doty S B, O'Brien S J, Warren R F. Thermal modification of collagen. Journal of shoulder and elbow surgery/American Shoulder and Elbow Surgeons . . . [et al. 1999; 8(4):339-344; Weiss R A, Weiss M A, Munavalli G, Beasley K L. Monopolar radiofrequency facial tightening: a retrospective analysis of efficacy and safety in over 600 treatments. J Drugs Dermatol. 2006; 5(8):707-712; Whipple T. From Mini-Invasive to Non-Invasive Treatment Using Monopolar Radiofrequency: The Next Orthopaedic Frontier. The Orthopedic clinics of North America. 2009; 40(4):531-535; Whipple T, Villegas D. Thermal Energy Fields by Non-invasive Monopolar Capacitive-Coupled RF: Temperatures Achieved and Histological Outcomes in Tendons and Ligaments. Richmond, Va.: Orthopaedic Research of Virginia; 2009; Wolf B R, Heiner A D, Albright J P, Nepola J V. Excessive radiofrequency application: effects on capsular tissue in an animal model. Journal of shoulder and elbow surgery/American Shoulder and Elbow Surgeons . . . [et al. 2005; 14(2):149-156; Wolf R S, Lemak L J. Thermal capsulorrhaphy in the treatment of multidirectional instability of the shoulder. J South Orthop Assoc. 2002; 11(2):102-109; Wright N T, Humphrey J D. Denaturation of collagen via heating: an irreversible rate process. Annu Rev Biomed Eng. 2002; 4:109-128; Yaar M, Gilchrest B A. Skin aging: postulated mechanisms and consequent changes in structure and function. Clin Geriatr Med. 2001; 17(4):617-630, v; Zelickson B D, Kist D, Bernstein E, Brown D B, Ksenzenko S, Burns J, Kilmer S, Mehregan D, Pope K. Histological and ultrastructural evaluation of the effects of a radiofrequency-based nonablative dermal remodeling device: a pilot study. Archives of dermatology. 2004; 140(2):204-209.

### Electrical Methods for Monitoring Tissue Treatment

In particular aspects herein, the invention comprises methods of monitoring a change in one or more structures or tissues (e.g., skin, a capsule, a vascular wall, a vaginal or urethral wall, etc.) by applying an AC potential between an active electrode and a return pad (passive electrode) to determine the variation in transmission of the applied electric field; exposing the tissue or collagen structures to treatment which could putatively alter the treated tissue (e.g., denature it); applying a de-novo AC potential and comparing the variation between the initial and the follow-up measurement.

This system can optionally be applied independently, or as part of a circuit providing immediate feedback to the system delivering the thermotherapy in such a way that this feedback loop can regulate the system's energy deliver to achieve a pre-determined end-point. In other words, electrical monitoring systems of the invention can be configured similarly to those systems using optical monitoring (e.g., in terms of regulation, automation, feedback, etc.).

This embodiment is based on the physical concept of electrical permittivity, which is a physical quantity that describes how an electric field affects and is effected by a dielectric medium (i.e., treated tissue as a dielectric medium) and is determined by the ability of a material to polarize in response to the field and thereby reduce the total electric field inside the material. Thus, permittivity relates to a material's ability to transmit (or “permit”) an electric field.

In short, to evaluate the extent of tissular changes, permittivity is measured by applying an AC potential between an active electrode and a return pad to determine the variation in transmission of the applied electric field.

In some aspects, the invention includes embodiments of methods of monitoring one or more change in structures (e.g., collagen) in a tissue through exposing the tissue to a first AC potential; measuring the permittivity of the exposed tissue; exposing the tissue to one or more treatments (which optionally or purportedly can alter tissular structures, thereby producing one or more treated tissues; exposing the treated tissue to a second AC potential; measuring a second permittivity of the exposed tissue; and comparing the first obtained permittivity value and the second obtained permittivity, thereby monitoring a change in the tissular structures. In such embodiments, the tissue can comprise a single tissular structure of layer or a first and at least a second tissular structure or layer which can be monitored simultaneously or sequentially. If there are two structures/layers to be monitored, one of such can be (and often typically is) closer to the surface of the tissue and/or closer to the point where treatment is being applied (e.g., RF energy). The tissular structure being monitored can comprise dermal collagen, mucosal collagen, synovial collagen, a tendon, a ligament, a fascia, or an aponeuourosis, etc. The tissue being monitored (the tissue comprising the structure such as collagen) can comprise skin, a fascia, an aponeurosis, a muscle, a tendon, a ligament, a capsule, a vascular wall, a nerve, a vaginal wall, an introitus, or a urethra, etc. In the various embodiments, the treatment can comprise application of physical energy, application of radio frequency waves, application of ultrasound, application of heat, application of cold, or application of a cosmeceutical. Also, in the various embodiments, the first and second AC potentials can be in the range of about 12 to about 300 volts; about 12 to about 48 volts; about 150 to about 300 volts or about 10 to about 50 mA, about 20 to about 40 mA and the duration of the various impulses can be between about 0.05 to about 10 msec. In some embodiments, comparing the AC potentials comprises comparing the voltages. Also, in some embodiments, the first and second AC potentials can be of different magnitude depending on the structure treated or under examination and the AC potential can be a square wave pulse or a sinusoid wave pulse and can be controlled in its intensity and/or duration.

In some embodiments of the invention, multiple active electrodes and/or return pads are provided. By changing the proximity of the electrodes and pads the effective measurement volume is varied, allowing for measurement in multiple tissue areas or to multiple tissue depths, enabling distinction between different structures within the tissue. In analogous manner to the optical methods also described herein (see above), such methods allow for separate monitoring of different structures in the tissue during a treatment. For example, the electric permittivity measured in shallow layers, such as the dermis, may be distinguished from the electric permittivity measured in a deeper structure such as a tendon or ligament being targeted for treatment.

In some aspects, the invention includes systems or devices for monitoring a change in one or more tissular structures. Such systems/devices can include: an AC potential source component; a detection/measuring component; and, a computer component programmed to: control the AC potential; deliver a first AC potential to the analyzed tissue; direct the detection/measurement component to measure the permittivity from the one or more tissular structures; deliver a second AC potential to the analyzed tissue; direct the detection/measurement component to measure the permittivity from the one or more tissular structures; and, compare the first permittivity value and the second permittivity value, thereby monitoring the changes in the one or more collagen structure. In the various systems/devices herein the tissue being monitored can comprise at least a tissular structure or a first and at least a second tissular structure. Also, the computer component in the systems/devices can be programmed to deliver the AC potential to expose the tissue to a first electrical charge and at least a second AC potential to establish a comparison of permittivity. The computer component can be programmed to differentiate changes in the permittivity of the tissular structure and, via a feed-back loop, to optionally control the system providing the thermotherapy or other therapeutic modality.

### Electrosurgical System and Methods for Generating a Wound Healing Response in Deep Tissues in a Non-Invasive Fashion

In some embodiments, the invention comprises an electrosurgical method for generating deep electric and thermal fields in tissues thus causing changes in the biochemical milieu and a wound healing response (WHR) in deep tissues in a noninvasive fashion, as well as devices for such methods. The embodiments can include monopolar direct coupled RF (mdRF), monopolar capacitive-coupled RF (mcRF) or bipolar RF methods for inducing changes in chemical milieu resulting in WHR, neuromodulation and neurolysis. Such embodiments can comprise: positioning an active electrode over the skin of a subject above or near-by to the targeted deep tissue, applying electromagnetic energy through the active electrode and having a return electrode in order to create deep energy and thermal fields resulting in changes in biochemical milieu and/or a thermal wound while avoiding damage to the skin and subcutaneous layers of tissue and resulting in stimulation of heat shock proteins and the expression of at least one mediator of the wound healing response cascade. It will again be appreciated that other aspects of the invention (e.g., treatment monitoring aspects, electrode design aspects, disposable tip electrodes, and contact sensing techniques) can, either separately or in any combination, be used with or in conjunction with the electrosurgical methods/devices herein.

In some embodiments, the invention comprises an electrosurgical method for generating a wound healing response in deep tissues in a noninvasive fashion. Such embodiments can comprise positioning an active electrode over the skin of a subject above or near-by to the targeted deep tissue; applying electromagnetic energy through the active electrode; and having a return electrode in order to create a deep electric and thermal field sufficient to generate a thermal wound resulting in stimulation of heat shock proteins and expression of at least one mediator of the wound healing response cascade. In such methods, the skin can be protected through controlled contact cooling generated, e.g., from an array of thermo-electrical coolers acting over a core member, the active electrode, to keep it from becoming too hot. See below. The electrosurgical methods can further comprise the creation of a de-novo wound in targeted tissue (e.g., a thermal wound which can result in the stimulation of elements of the healing response such as heat shock proteins, cytokines, etc.). In the various methods herein, the deeper tissue can include an area of tissue that would benefit from an active wound healing response. Such areas can include, e.g., a tendon, a ligament, a fascia, an aponeurosis, a capsule, a nerve fiber, a vessel, a muscle, a bone, or any connective tissue, etc. Furthermore, the methods can comprise inducing coagulation of connective tissue.

In some embodiments, the invention comprises an electrosurgical method for generating electric and thermal fields in deep tissues in a noninvasive fashion comprising: positioning an active electrode over the skin of a subject above or nearby to the targeted deep tissue; applying electromagnetic energy through the active electrode; and having a return electrode in order to create a deep electric and thermal field sufficient to modify local biochemical milieu in nervous tissue. In such embodiments, altering the local biochemical milieu can comprise changes to the expression of neuropathic pain markers or neuropathic pain mediators; changing the expression of neuropathic pain markers and mediators, wherein at least one pain marker is substance P; changing the expression of neuropathic pain markers and mediators, wherein at least one pain marker is Glial fibrillary acidic protein (GFAP); changing the expression of neuropathic pain markers and mediators, wherein at least one pain mediator is neurokinin-1 receptors changing the expression of neuropathic pain markers and mediators, wherein at least one pain mediator is calcitonin gene related peptide (CGRP); or changing the expression of mitogen-activated protein kinases (MAPK).

In some embodiments, the invention comprises an electrosurgical method for generating a wound healing response in deep tissues in a noninvasive fashion comprising: positioning an active electrode over the skin of a subject above or nearby to the targeted deep tissue; applying electromagnetic energy through the active electrode; and having a return electrode in order to create a deep electric and thermal field sufficient to generate a thermal wound resulting in stimulation of heat shock proteins and expression of at least one mediator of the wound healing response cascade. Such methods can further comprise inducing angiogenesis in the targeted tissue. Also, in such methods, the active electrode can be displaced to cover a volume of underlying tissue and the active electrode can be pulsed (e.g., from about 10 msec to about 500 seconds) or can be continuous.

These present embodiments pertain to electrosurgical systems and methods for treating tissue, in particular, generating electric and thermal fields in deep tissues in a noninvasive fashion comprising: positioning an active electrode over the skin above or nearby to the targeted deep tissue; applying electromagnetic energy through the active electrode; and having a return electrode in order to create a deep electric and thermal field sufficient to modify local biochemical milieu in nervous tissue. Again, some embodiments can comprise one or more aspect of other embodiments herein too. For example, the embodiments within this section can comprise the embodiments described below of “electrode design to avoid edge and corner effects”, “disposable tip for directly coupled electrode”, “capacitive contact sensing methods”, etc.

It will be appreciated that the invention also includes devices for such methods herein. Furthermore, the embodiments of the invention can involve noninvasive (or only minimally invasive) interventions that can be used to therapeutically and/or prophylactically treat subjects. Previous interventions/treatments relied on surgery or needle access to reach tissues to be targeted/treated. In contrast, by using mcRF or mdRF, the present invention avoids the need for surgery or needle access to reach tissues to be treated. Also, other prior noninvasive techniques were unable to provide the level of temperature needed to bring about change in the biochemical mediators of pain and/or to bring about wound healing responses. Again, in contrast, the present invention does have the proper temperature regulating ability to actually modify pain mediators and/or bring about wound healing in a noninvasive manor. Thus, the various embodiments comprising combinations of upper layer tissue cooling and deep structure tissue heating can be used to create wound healing responses, modify pain mediators, etc. Various embodiments can be used for cold neuromodulation, and can be used to selectively target particular structures and/or tissues for thermal treatment (e.g., by controlling temperatures in either targeted or untargeted structures/tissues), etc.

Embodiments of the present invention describe a method of “cold neuromodulation” in which the tissues can be exposed to high current density, without the need for direct contact between the electrode and the tissues targeted for neuromodulation, while the thermal impact can be minimized or completely counteracted. One benefit of such is the ability to avoid the invasiveness of some of the current neuromodulation methods, and avoid the strong limitations of energy delivery (and therefore effectiveness) imposed on non-invasive neuromodulation methods.

In some embodiments, the invention includes a method of providing neuromodulation of tissues in which high current density is generated and transmitted through non-targeted tissues and the thermal energy generated in tissue is counteracted by an active cooling system, wherein the flow of current can be continuous.

In some embodiments, the invention includes a method of providing neuromodulation of tissues in which high current density is generated and transmitted through non-targeted tissues and the thermal energy generated in tissue is counteracted by an active cooling system wherein the flow of current can be pulsed.

Other embodiments include a method of providing neuromodulation of tissues in which high current density is generated and transmitted through non-targeted tissues and the thermal energy generated in tissue is counteracted by a passive cooling system wherein the flow of current can be continuous.

Yet other embodiments include a method of providing neuromodulation of tissues in which high current density is generated and transmitted through non-targeted tissues and the thermal energy generated in tissue is counteracted by a passive cooling system wherein the flow of current can be pulsed. Further discussion of cooling embodiments is presented below.

mcRF for the Treatment of Pain

Monopolar capacitive-coupled RF (mcRF) is a new and unique modality of RF allowing the delivery of high currents to deep tissues in a totally non-invasive fashion in contradistinction with current invasive alternatives that require anesthesia or intravenous sedation and the use of fluoroscopy and expose patients and medical personnel to significant risks. mcRF creates a deep-penetrating volumetric electric field that resembles the mechanism of action of pRF and is capable of volumetric heating with temperatures that could reach above those generated by pRF and below those achieved with cRF. At high energy outputs, mcRF is capable of non-invasive electrocoagulation.

mcRF was introduced to the field of plastic surgery with demonstrated safety and efficacy. Recently, mcRF was introduced to the field of orthopaedics (AT2™ System, Alpha Orthopaedics, Hayward, Calif.) as a tool for non-invasive electrocoagulation of tissue. Current data suggest that the mcRF technology has significant clinical value when treating pain associated with overuse injuries of the musculoskeletal system.

Facet and SI Joints

Current clinical experience with mcRF indicates that the technology has the potential to become the noninvasive tool of choice when treating patients with chronic pain originating from the zygapophyseal and sacroiliac joints, reserving invasive alternatives for the treatment of recalcitrant cases.

Peripheral Nerves Treatment

mcRF has a demonstrated antinociceptive effect when used in treating musculoskeletal conditions such as tennis elbow, acute and even chronic ankle sprains. It is theorized that this antinociceptive effect is the result of “resetting” of the afferent fibers. Initial clinical experiences (case reports) suggest that non-invasive mcRF may have a very positive impact in peripheral afferent nerves.

### Technical Considerations of the Embodiments

In some embodiments, the methods and devices comprise use of monopolar direct coupled RF (mdRF). mdRF creates an electromagnetic energy field beneath the application electrode by alternating current between the active electrode and the return pad (passive electrode). In addition to the electric field, resistive heating of tissue results, which is the actual source of heat, rather than the electrode itself.

Temperatures achieved at targeted structures depend on treatment technique and the user's determined energy output. The system can automatically adjust the intensity of the output based on the patient's tissue impedance readings. Resulting temperatures may remain within physiological levels or reach supra-physiological points that are capable of non-invasive electrocoagulation, without compromising the integrity of the skin that is in direct contact with the active electrode, thereby preserving shallow structures. Depending on electrode design, the electrical field and therefore thermal effect (penetrating depth) of mdRF energy is deeper and wider than any light-based energy modalities.

The elimination of heat depends on two factors: the conductivity and vascularization of the tissue (blood flow carries heat away from the heated area). Targeted structures (i.e., nerves, tendons, ligaments, and fascias) are poorly vascularized when compared with skin, therefore allowing for differential heat retention that favors the desired outcome.

Treatment of Tissue Using mdRF

In the mdRF embodiments of the invention, mdRF is used to generate electric fields in treated tissue. mdRF is capable of combining neuromodulation (traditionally offered by pRF) and neurolysis (traditionally offered by cRF) with the significant advantage of being non-invasive.

If the clinician's judgment calls for neuromodulation only, mdRF at a low setting will expose the tissue to a current field as a way to alter the pain signal's pathway. Temperatures generated by the electric field are then left to dissipate between pulses, thereby keeping overall temperature below 45° C., since changes are thought to be reversible up to this temperature.

If the clinician's judgment calls for neurolysis, then mdRF at a higher setting can reach the desired elevated temperatures between 45° C. and 55° C. impacting small fibers without ablating tissue. The axons associated with nociceptors are lightly myelinated or, more commonly, unmyelinated (type Aδ group or C fibers); non-ablative temperatures have a selective effect on small unmyelinated nerve fibers. mdRF specifically aims to expose neural structures to non-ablative temperatures, preventing the deafferentation or denervation sequelae and leaving large fibers relatively intact.

Temperatures above 55° C. are also achievable, resulting in tissue coagulation and eventually ablation. Coagulation of protein occurs at temperatures around 60° C. This is typically not favored; in fact, pain specialists prefer non-ablative techniques when working with complex structures such as DRG.

If the clinician's judgment calls for a combination of neuromodulation and neurolysis, mdRF at a medium setting can generate the desired electric field as well as temperatures capable of inducing physical changes in neural structures.

Neutralization of Pain Mediators by mdRF

The electric field delivered by non-invasive mdRF can reduce the levels of Substance P by reducing or suppressing C-fiber nerves or causing these nerves to be more tolerant. It is proposed that mdRF may have an antagonist effect to NK1 and possibly to NK2. These effects can be exploited to down-regulate the sensitivity of pain sensors (nociceptors) resulting in an analgesic effect.

In fact, chronic pain can lead to local pain receptors becoming over-sensitized. These over-sensitive receptors then further continue the cycle by secreting pain-mediating neuropeptides (e.g. substance P, NK1, GFAP, CGRP) in response to even the smallest stimuli. This vicious cycle is a frequent contributor to chronic pain and inflammation syndromes that are resistant to therapy. Other factors may also play a role in the impact of mdRF such as changes in ion channels and pH levels.

Resetting of Aberrant Afferent Input in Functional Ankle Instability (FAI) by mdRF

Functional Ankle Instability (FAI) is related to the neuromuscular control of the ankle and is characterized by impaired joint kinesthesia and altered muscle recruitment patterns resulting in subject's feeling of the ankle giving way. It is postulated that the electric field delivered by non-invasive mdRF “reset” these afferent fibers (Neuromodulation) allowing for a rapid recovery as new—non-pathological—afferent signals are sent to the Central Nervous System (CNS). Moreover, manipulation of wound electric fields affects wound healing in vivo. Electric stimulation triggers activation of Src and inositol-phospholipid signaling, which polarizes in the direction of cell migration. Notably, genetic disruption of phosphatidylinositol-3-OH kinase-gamma (PI (3) Kgamma) decreases electric-field-induced signaling and abolishes directed movements of healing epithelium in response to electric signals.

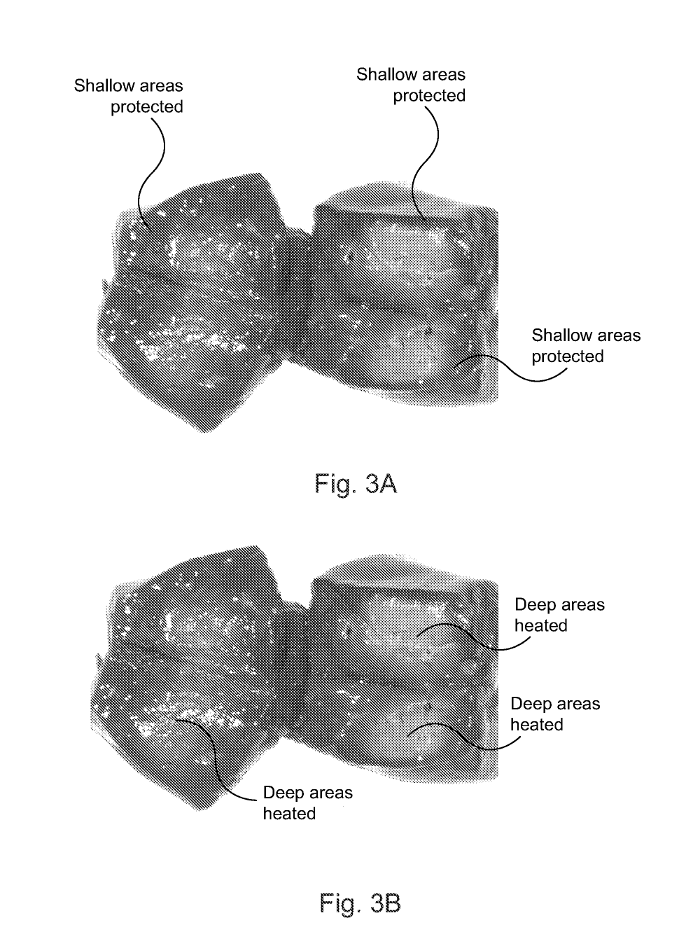

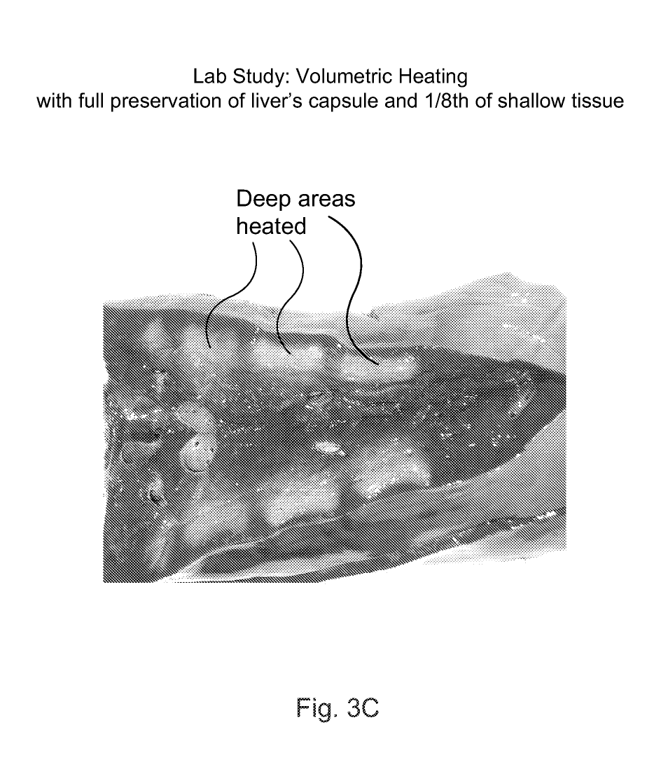

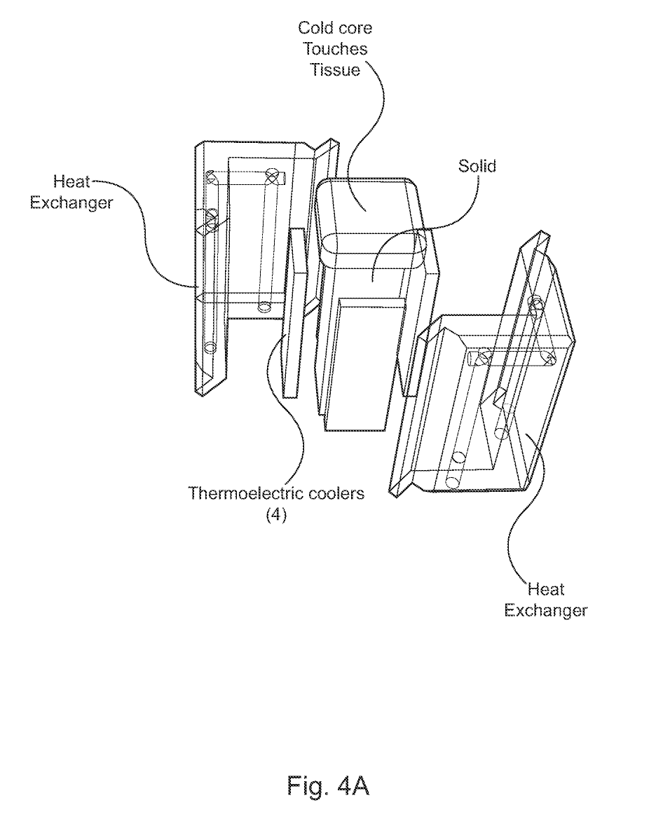

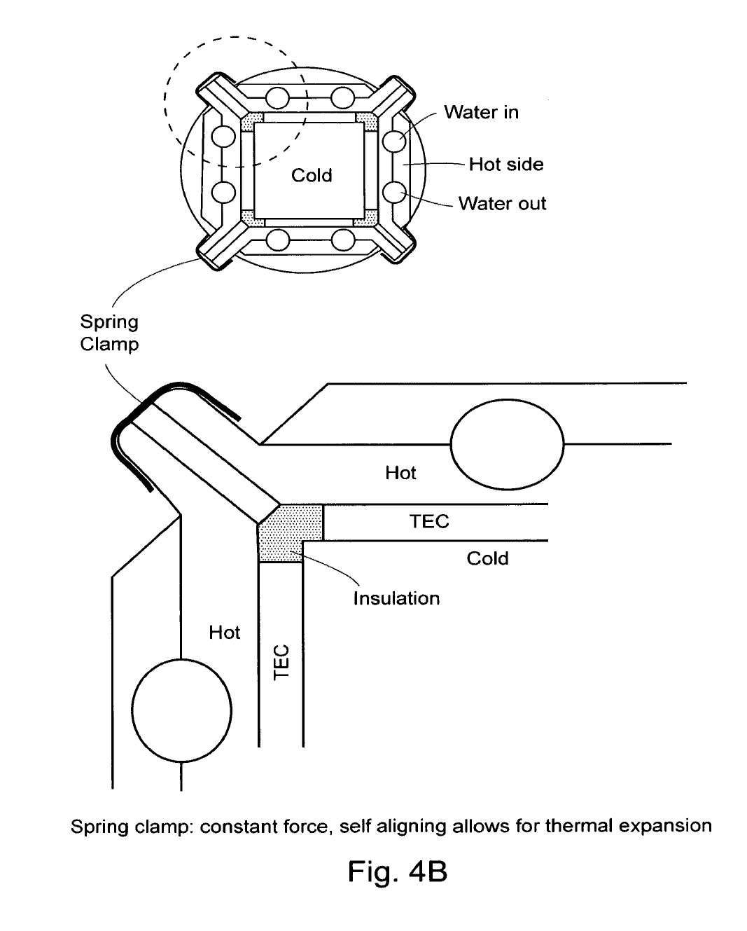

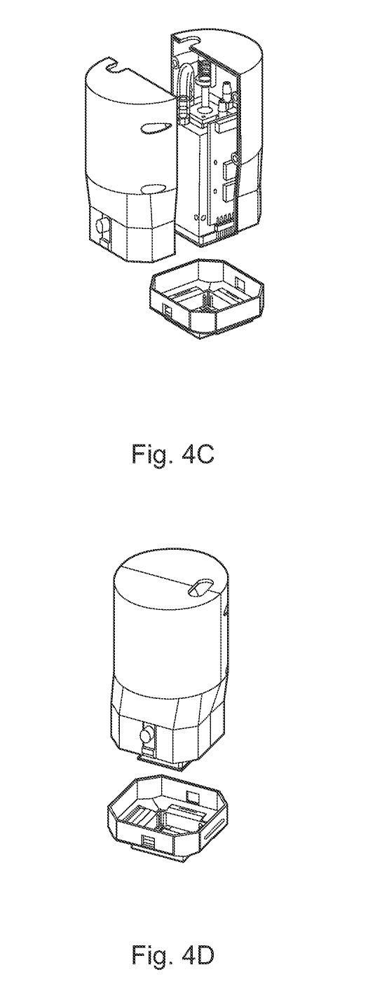

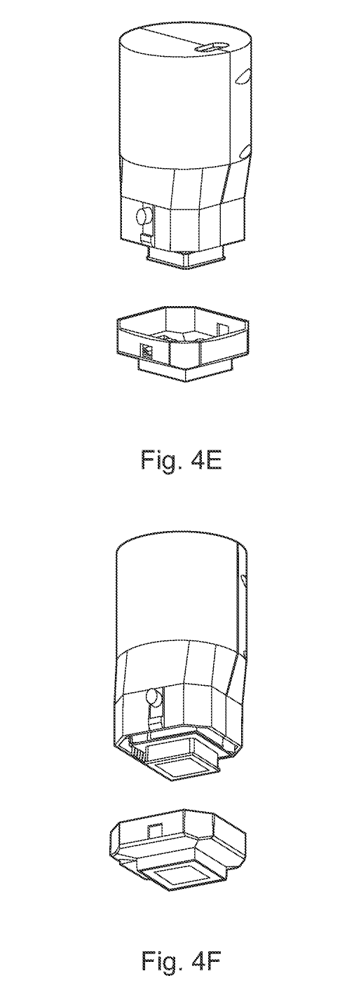

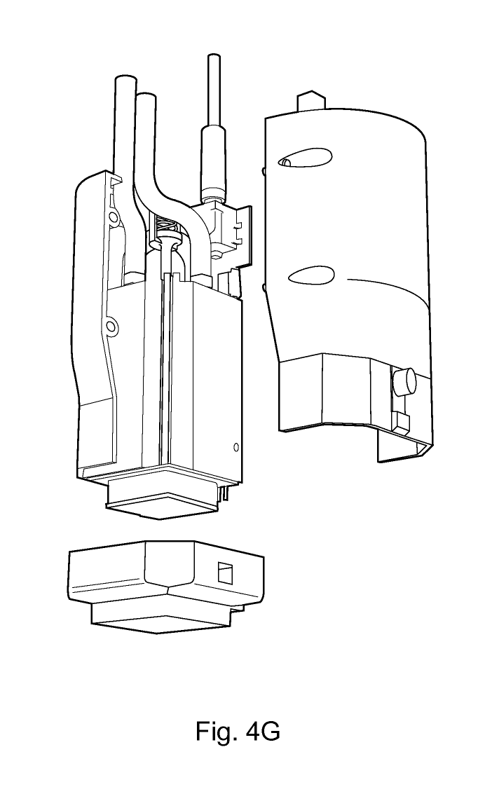

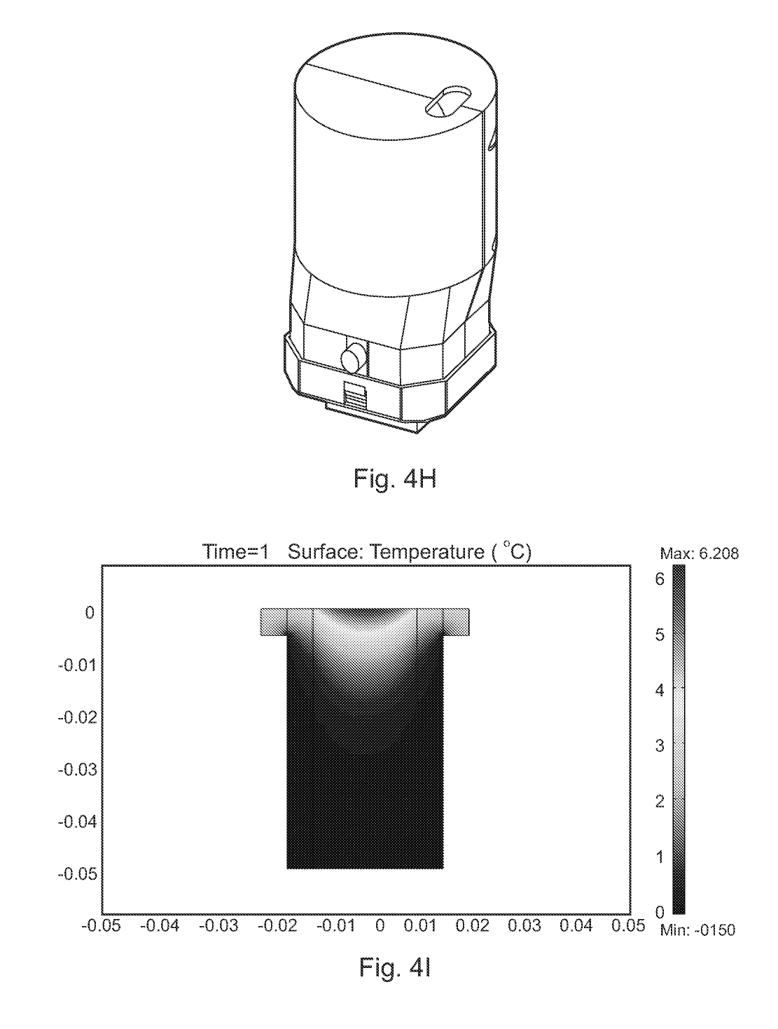

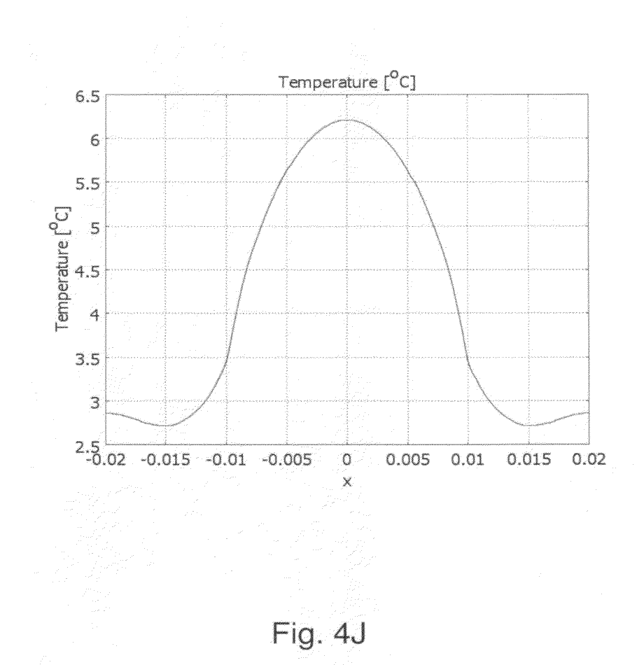

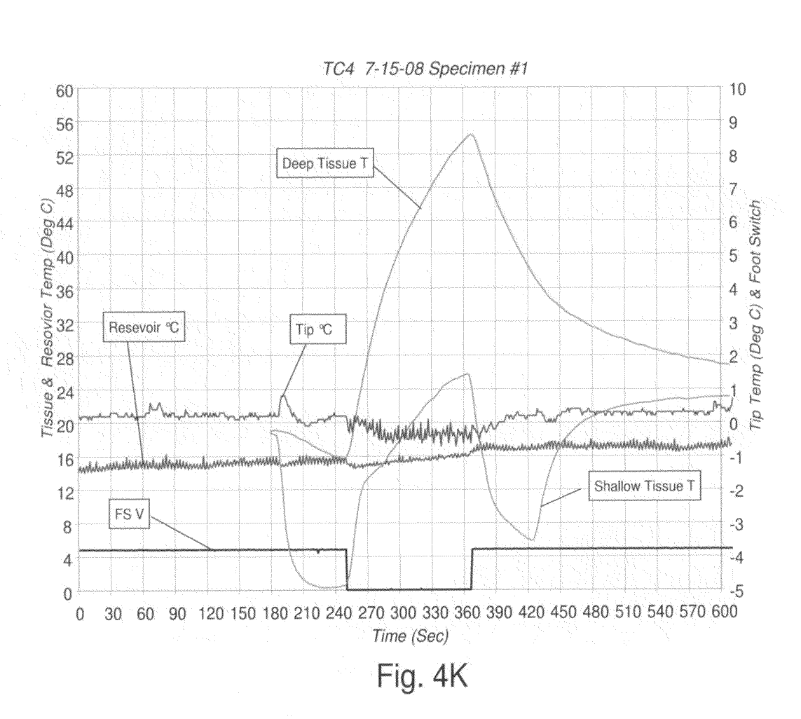

FIGS. 3a-c illustrate the controlled deep tissue thermoregulation (without tissue surface heating) capable of exemplary embodiments of the invention using mdRF. As can be seen, internal areas of the tissue (here liver) were heated, with corresponding internal tissue changes, while the surfaces of the tissues were not adversely affected. Additional examples of deep tissue thermo-treatment (without surface heating) can be seen in FIG. 4k below. To obtain these outcomes, an mdRF generator coupled to a copper electrode with a thin ceramic plate around the periphery of the patient interface was used. The ceramic plate was cooled but did not allow an electrically conductive path, thus creating a cooling border around the treatment site (described further below). The electrode was in direct contact with the tissue at all times during the treatment. The change in color from dark red of a normal liver tissue to brown indicates that the elevation in temperature was enough to change tissular properties while the tissue in direct contact with the electrode remained unchanged. The size of each individual tissular block changed was about 2 by 2 by 2 cms. This volumetric change has direct application in the medical field in areas where a variety of changes may be desirable from induction of the wound healing response, contraction of collagenous molecules, ablation of tissue, and the like. A correlation between temperatures achieved and histological characteristics of tissue can be established and utilized to define the targeted outcome. In the example provided in FIGS. 3a-c, total denaturation of liver tissue was achieved at temperatures in the range of 50 to 55° C.

Various references useful for the embodiment, as well as optionally for other sections and embodiments herein, include, e.g., Hertel J. Functional Anatomy, Pathomechanics, and Pathophysiology of Lateral Ankle Instability. J Athl Train. 2002; 37(4):364-375; Marshall P W, McKee A D, Murphy B A. Impaired trunk and ankle stability in subjects with functional ankle instability. Med Sci Sports Exerc. 2009; 41(8):1549-1557; McCaig C D, Rajnicek A M, Song B, Zhao M. Controlling cell behavior electrically: current views and future potential. Physiol Rev. 2005; 85(3):943-978; Shah J P, Danoff J V, Desai M J, Parikh S, Nakamura L Y, Phillips T M, Gerber L H. Biochemicals associated with pain and inflammation are elevated in sites near to and remote from active myofascial trigger points. Arch Phys Med Rehabil. 2008; 89(1):16-23; Shah J P, Gilliams E A. Uncovering the biochemical milieu of myofascial trigger points using in vivo microdialysis: an application of muscle pain concepts to myofascial pain syndrome. J Bodyw Mov Ther. 2008; 12(4):371-384; Yeung M S, Chan K M, So C H, Yuan W Y. An epidemiological survey on ankle sprain. Br J Sports Med. 1994; 28(2):112-116; Zhao M, Song B, Pu J, Wada T, Reid B, Tai G, Wang F, Guo A, Walczysko P, Gu Y, Sasaki T, Suzuki A, Forrester J V, Bourne H R, Devreotes P N, McCaig C D, Penninger J M. Electrical signals control wound healing through phophatidylinositol-3-OH kinase-gamma and PTEN. Nature. 2006; 442(7101):457-460.

### Electrode Design to Avoid Edge and Corner Effects

The embodiments described herein provide devices to avoid edge and corner burns on electrodes used to deliver energy to tissue (e.g., for RF treatment of tissues, etc.).

RF energy is delivered through electrodes that couple the energy to the chosen tissue with therapeutic purposes. The anticipated outcome is a rise in tissue temperature to a predictable level, resulting in a range of histological effects from temporary stimulation of local circulation through ablation of the tissue. See throughout. Unfortunately, in many prior uses, RF energy conducted through electrodes was concentrated at the edges of the electrode, as opposed to having a uniform distribution over the surface. This concentration of RF energy or edge effect results in undesirable burns to the exposed tissue.

In prior attempts to avoid the edge effect, different sets of dielectrics have been used in an attempt to achieve a more uniform conductivity of the energy into the tissue. The present embodiments, however, provide a new electrode that overcomes the edge effect by one or more of the following: providing peripheral cooling to the electrode wherein electrode edges are cooler than the rest of the electrode surface “peripheral preferential cooling”; providing peripheral cooling outside of the area of electrical energy transfer; regulating thermal energy transfer properties from the electrode to the underlying tissue by either concentric rings of different metals or metal alloys, in which the thermal exchange between the electrode and underlying tissue is higher in the periphery and lower in the center of the electrode by making the outer ring of highly thermally conductive material whereas the inner ring will have relatively lower thermal conductivity (intermediate rings may also be provided, having intermediate thermal energy transfer properties so as to create a gradient); regulating electrical energy transfer properties from the electrode to the underlying tissue by either concentric rings of different metals or metal alloys, in which the electrical exchange between the electrode and underlying tissue is higher in the center and lower in the periphery of the electrode by making the outer ring of poorly electrical conductive material whereas the inner ring will have relatively higher electrical conductivity (intermediate rings may also be provided, having intermediate electrical energy transfer properties such as to create a gradient); through custom alloy(s) cast such that when exposed to electrical energy and/or thermal energy, a temperature gradient is created by the variation in the alloys' thermal resistance; through custom alloy(s) cast such that when exposed to electrical energy and or thermal energy, a temperature gradient is created by the variation in the alloys' electrical resistance; through use of a semiconductor (a solid material with electrical conductivity in between that of a conductor and that of an insulator) that can vary either permanently or dynamically; through use of a variable thickness of the electrode in which thermal resistance is modulated by thickness of material as opposed to its composition; through a variable thickness on the electrode in which electrical resistance is modulated by thickness of material as opposed to its composition; or through any combination of such elements.

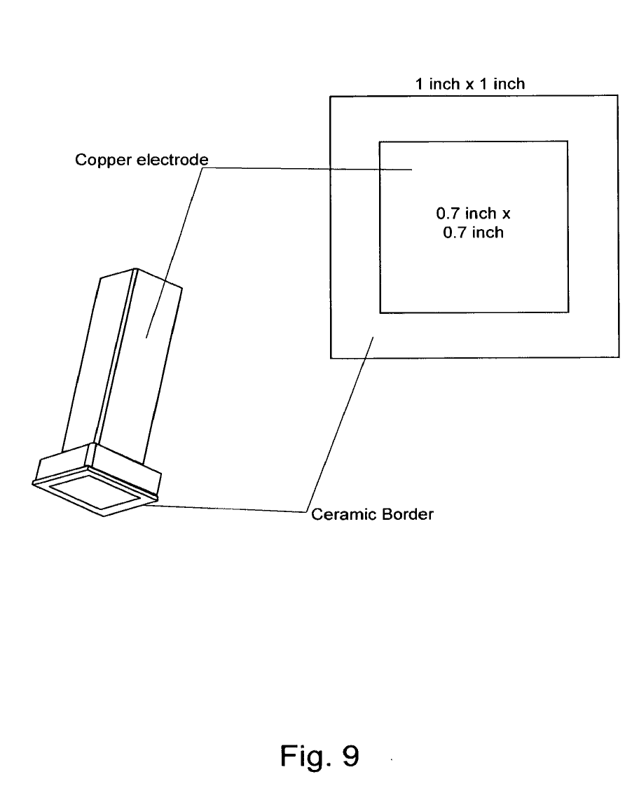

An example of one embodiment is comprised of a copper electrode with a thin ceramic plate around the periphery of the patient interface. The ceramic plate is cooled but does not allow an electrically conductive path, thus creating a cooling border around the treatment site. See FIG. 9.

In some embodiments, the invention includes an apparatus comprising a skirt thermoelectric cooling device that is attached circumferentially to an electrode periphery that mitigates electrode edge effect in tissue (to which the electrode is applied) wherein the skirt is attached to either a directly coupled or capacitive coupled electrode. Use of such skirt thermoelectric cooling (TEC) device reduces electromagnetic heating of tissue beyond the periphery of the electrode while maintaining cooling and also allows for the elimination of the use of chlorofluorocarbon (CFC) gases for cooling.

In some embodiments, the invention includes an apparatus wherein the reduction of electrode edge effect is accomplished with a transitional alloy electrode. The electrode can be doped with an alloy constituent that modifies the electrical resistance of the electrode. With some embodiments, the electrode becomes more electrically resistive towards the periphery of the electrode.

In some embodiments, in combination with a skirt thermoelectric cooling device, the mitigation of an electrode edge effect can be achieved with a composite electrode that is more thermally conductive at the periphery in a controlled thermal conductivity (e.g., insulating layers).

Tables 1 and 2 present comparative thermal conductivity and electrical resistivity of a number of materials, any of which are optionally comprised as part of the devices herein.

TABLE 1

| | Thermal | |
|---|---|---|
| | conductivity | Thermal |
| | (cal/sec)/(cm² | conductivity |
| Material | C./cm) | (W/m K)* |
| Diamond | . . . | 1000 |
| Silver | 1.01 | 406 |
| Copper | 0.99 | 385 |
| Gold | . . . | 314 |
| Brass | . . . | 109 |
| Aluminum | 0.5 | 205 |
| Iron | 0.163 | 79.5 |
| Steel | . . . | 50.2 |
| Lead | 0.083 | 34.7 |
| Mercury | . . . | 8.3 |
| Ice | 0.005 | 1.6 |
| Glass, ordinary | 0.0025 | 0.8 |
| Concrete | 0.002 | 0.8 |
| Water at 20° C. | 0.0014 | 0.6 |
| Asbestos | 0.0004 | 0.08 |
| Snow (dry) | 0.00026 | . . . |
| Fiberglass | 0.00015 | 0.04 |
| Brick, insulating | . . . | 0.15 |
| Brick, red | . . . | 0.6 |
| Cork board | 0.00011 | 0.04 |
| Wool felt | 0.0001 | 0.04 |
| Rock wool | . . . | 0.04 |
| Polystyrene (styrofoam) | . . . | 0.033 |
| Polyurethane | . . . | 0.02 |
| Wood | 0.0001 | 0.12-0.04 |
| Air at 0° C. | 0.000057 | 0.024 |
| Helium (20° C.) | . . . | 0.138 |
| Hydrogen(20° C.) | . . . | 0.172 |
| Nitrogen(20° C.) | . . . | 0.0234 |
| Oxygen(20° C.) | . . . | 0.0238 |
| Silica aerogel | . . . | 0.003 |

TABLE 2

| | | | Temperature | |
|---|---|---|---|---|
| | Resistivity | | coefficient per | Conductivity |
| Material | Ohm · m | | degree C. | S/m |
| Silver | 1.59 | ×10\hat{ }−8 | 0.0061 | 6.29 |
| Copper | 1.68 | ×10\hat{ }−8 | 0.0068 | 5.95 |
| Aluminum | 2.65 | ×10\hat{ }−8 | 0.00429 | 3.77 |
| Tungsten | 5.6 | ×10\hat{ }−8 | 0.0045 | 1.79 |
| Iron | 9.71 | ×10\hat{ }−8 | 0.00651 | 1.03 |
| Platinum | 10.6 | ×10\hat{ }−8 | 0.003927 | 0.943 |
| Manganin | 48.2 | ×10\hat{ }−8 | 0.000002 | 0.207 |
| Lead | 22 | ×10\hat{ }−8 | . . . | 0.45 |
| Mercury | 98 | ×10\hat{ }−8 | 0.0009 | 0.1 |
| Nichrome | 100 | ×10\hat{ }−8 | 0.0004 | 0.1 |
| (Ni, Fe, Cr alloy) | | | | |
| Constantan | 49 | ×10\hat{ }−8 | . . . | 0.2 |
| Carbon* | Mar-60 | ×10\hat{ }−5 | −0.0005 | . . . |
| (graphite) | | | | |
| Germanium* | 1-500 | ×10\hat{ }−3 | −0.05 | . . . |
| Silicon* | 0.1-60 | . . . | −0.07 | . . . |
| Glass | 1-10000 | ×10\hat{ }9 | . . . | . . . |
| Quartz | 7.5 | ×10\hat{ }17 | . . . | . . . |
| (fused) | | | | |
| Hard rubber | 1-100 | ×10\hat{ }13 | . . . | . . . |

The depth of tissue cooling with a skirt thermoelectric cooling device is both temperature and time dependent. A steady state of surface tissue cooling is reached where the depth of cooling is determined by the specified temperature of the thermoelectric skirt. Once a steady state is reached, the skirt electrode device can be kept continuously in contact with the tissue surface while moving the device without lifting or placing for the next application. The thermal dose will be determined by the speed that the device is advanced across the tissue surface. A slower traverse of the device across the tissue surface by the practitioner will provide a greater thermal dose to the subjacent target tissue. Likewise, a faster traverse will reduce the thermal dose to the target tissue.

In some embodiment, software feedback control of RF output can be provided by thermistors placed on the perimeter of the skirt electrode device to monitor tissue surface temperature. Reaching a high temperature limit thus activates a software controlled reduction of the RF output until temperature monitoring of the surface is within a safe thermal profile.

In yet another embodiment, software feedback control of RF output can be provided by thermistors placed on the perimeter of the skirt electrode device to monitor tissue surface temperature. Reaching a low temperature limit activates a software controlled reduction in the thermoelectric cooling and/or increase in RF output until temperature monitoring of the surface is within a safe thermal profile

In different embodiments, the apparatus comprises an electrode to conduct energy (e.g., RF energy delivered in either monopolar or bipolar fashion) to tissue wherein the electrode is used in direct contact with tissue. The electrode can be, e.g., round or polygonal in shape and have a cooling system provided from the periphery. In such cooling system, thermoelectric coolers can be attached to the periphery (e.g., parallel to the electrode surface or perpendicular to the electrode surface). The thermoelectric coolers can be at different angles to the electrode surface. In the embodiments, the area cooled can exceed the area of energy transfer. Also, concentric layers of materials can be laid in such a way that thermal transfer is maximum at the edges and minimum at the center or wherein concentric layers of materials are laid in such a way that electrical energy transfer is maximum at the center and minimum at the edges. Furthermore, the different layers can be electrically isolated from each other. In some embodiments, different radiofrequency generators can drive different levels of energy through the different layers so as to create a gradient of electrical energy transfer. Some embodiments can comprise a variable thickness dielectric material which provides power attenuation wherein electrical energy transfer is higher at the center of the electrode and lower at its periphery. In some embodiments, a custom cast alloy can be laid in such a way that electrical energy transfer is higher at the edges and maximum at the center. In some embodiments, the first layer of the concentric layers can be made out of, e.g., silver, the second of, e.g., cooper, the third of, e.g., aluminum, and the fourth of, e.g., iron. Also, in some embodiments, the concentric layers can have different layers made out of electrically conductive materials with a variety of thermal conductivity properties and a variety of electrical conductive properties. The electrode in various embodiments can include wherein a custom cast alloy is laid in such a way that thermal transfer is maximum at the edges and minimum at the center. Also, the electrode is optionally wherein variable thickness is used to control thermal transfer

In some embodiments, the apparatus comprises a cooling system comprised of a primary and a secondary subsystem. Such subsystems can optionally be thermally coupled through a fluid medium. Optionally, the primary subsystem precisely regulates the tip temperature while the secondary subsystem can optionally utilize TECs (active system). The secondary subsystem can optionally release heat to the environment through a radiator (passive system) and can optionally regulate the fluid temperature to a set point or less than or equal to a set point. In the various embodiments, the primary TECs can be electively driven to cool or heat in response to the varying RF load.

FIG. 4a shows an exploded view of an implementation of an electrode employing a TEC. The cold core is the electrode and also the cold (cooling) element of the invention. This core can be surrounded by the TECs on all four sides, with the cold sides of the TECs facing the core and the hot sides facing the heat exchangers. The heat exchangers can be thermally coupled by a fluid circulating back to a console (not shown), where a secondary cooling system of similar construction can be used to remove the heat and discharge to the air. The TECs can be reversible, so that when the flow of current is reversed, the hot and cold sides are reversed. This allows for precise and quick regulation of the temperature. FIG. 4b shows an exemplary apparatus comprising an electrode and surrounding TECs. FIGS. 4c-4h show an example embodiment for clinical use comprising a hand piece for packaging the electrode and a disposable element electrode cover.

The thermal properties of one exemplary embodiment of the electrode cooling system were numerically modeled, and the predicted thermal profile across the face of the electrode is summarized in FIG. 4i. The temperature profile across the electrode surface, shown in FIG. 4j, demonstrates the concentration of the cooling effect around the edges of the electrode, where RF energy concentration will be the greatest. FIG. 4k shows an example of a treatment profile where the shallower tissue temperature is clearly below the necrosis level, whereas the deeper (target region) is at therapeutic temperatures. As illustrated in FIG. 4k, a clinically significant temperature differential is achieved between the upper areas of tissue (non-targeted) vs. deep-targeted zone. Temperatures were monitored by inserting fluoroptic sensors in the tissues treated and followed over 10 minutes. A clear visual demonstration of the ability of the invention to provide uniform heating at predictable depths while keeping surface tissues unheated, is provided in FIGS. 3a-c, as discussed further above.

It will be appreciated that the various temperature controlled electrode aspects herein can be connected to various other components when used, e.g., computer or processing components, fluid flow components, etc. Computer or processing components can optionally control one or more of, e.g., the RF application, action of the TEC, etc. Monitoring embodiments, e.g., as detailed herein, can also optionally be used concurrently or along with the temperature controlled electrodes herein in order to monitor tissue/collagen content and/or status.

Various references useful for the embodiment, as well as optionally for other sections and embodiments herein, include, e.g., Allain, J. C.; Le Lous, M.; Bazin, S.; Bailey, A. J.; and Delaunay, A.: Isometric tension developed during heating of collagenous tissues. Relationships with collagen cross-linking. Biochim Biophys Acta, 533(1): 147-55, 1978; Allain, J. C.; Le Lous, M.; Cohen, S.; Bazin, S.; and Maroteaux, P.: Isometric tensions developed during the hydrothermal swelling of rat skin. Connect Tissue Res, 7(3): 127-33, 1980; Arnoczky, S. P., and Aksan, A.: Thermal modification of connective tissues: basic science considerations and clinical implications. Instr Course Lect, 50: 3-11, 2001; Avitall, B. et al.: Physics and engineering of transcatheter cardiac tissue ablation. J Am Coll Cardiol, 22(3): 921-32, 1993; Avitall, B.; Lafontaine, D.; Rozmus, G.; Adoni, N.; Le, K. M.; Dehnee, A.; and Urbonas, A.: The safety and efficacy of multiple consecutive cryo lesions in canine pulmonary veins-left atrial junction. Heart Rhythm, 1(2): 203-9, 2004; Avitall, B.; Urbonas, A.; Urboniene, D.; Rozmus, G.; and Helms, R.: Linear lesions provide protection from atrial fibrillation induction with rapid atrial pacing. J Cardiovasc Electrophysiol, 13(5): 455-62, 2002; Brodkey, J. S.; Miyazaki, Y.; Ervin, F. R.; and Mark, V. H.: Reversible Heat Lesions With Radiofrequency Current. A Method Of Stereotactic Localization. J Neurosurg, 21: 49-53, 1964; Chen, S. S.; Wright, N. T.; and Humphrey, J. D.: Heat-induced changes in the mechanics of a collagenous tissue: isothermal, isotonic shrinkage. J Biomech Eng, 120(3): 382-8, 1998; Cooper, T. E., and Trezek, G. J.: Correlation of thermal properties of some human tissue with water content. Aerosp Med, 42(1): 24-7, 1971; Cosman, E. R.; Nashold, B. S.; and Ovelman-Levitt, J.: Theoretical aspects of radiofrequency lesions in the dorsal root entry zone. Neurosurgery, 15(6): 945-50, 1984; Daoud, E., and Morady, F.: Catheter ablation of ventricular tachycardia. Curr Opin Cardiol, 10(1): 21-5, 1995; Hayashi, K.; Hecht, P.; Thabit, G., 3rd; Peters, D. M.; Vanderby, R., Jr.; Cooley, A. J.; Fanton, G. S.; Orwin, J. F.; and Markel, M. D.: The biologic response to laser thermal modification in an in vivo sheep model. Clin Orthop Relat Res, (373): 265-76, 2000; Hayashi, K.; Markel, M. D.; Thabit, G., 3rd; Bogdanske, J. J.; and Thielke, R. J.: The effect of nonablative laser energy on joint capsular properties. An in vitro mechanical study using a rabbit model. Am J Sports Med, 23(4): 482-7, 1995; Hayashi, K.; Peters, D. M.; Thabit, G., 3rd; Hecht, P.; Vanderby, R., Jr.; Fanton, G. S.; and Markel, M. D.: The mechanism of joint capsule thermal modification in an in-vitro sheep model. Clin Orthop Relat Res, (370): 236-49, 2000; Hayashi, K.; Thabit, G., 3rd; Bogdanske, J. J.; Mascio, L. N.; and Markel, M. D.: The effect of nonablative laser energy on the ultrastructure of joint capsular collagen. Arthroscopy, 12(4): 474-81, 1996; Lesh, M. D.: Interventional electrophysiology—state-of-the-art 1993. Am Heart J, 126(3 Pt 1): 686-98, 1993; Moraci, A.; Buonaiuto, C.; Punzo, A.; Parlato, C.; and Amalfi, R.: Trigeminal neuralgia treated by percutaneous thermocoagulation. Comparative analysis of percutaneous thermocoagulation and other surgical procedures. Neurochirurgia (Stuttg), 35(2): 48-53, 1992; Saxon, L. A.; Kalman, J. M.; Olgin, J. E.; Scheinman, M. M.; Lee, R. J.; and Lesh, M. D.: Results of radiofrequency catheter ablation for atrial flutter. Am J Cardiol, 77(11): 1014-6, 1996; Seegenschmiedt, M. H., and Sauer, R.: The current role of interstitial thermo-radiotherapy. Strahlenther Onkol, 168(3): 119-40, 1992; Sweet, W. H., and Wepsic, J. G.: Controlled thermocoagulation of trigeminal ganglion and rootlets for differential destruction of pain fibers. 1. Trigeminal neuralgia. J Neurosurg, 40(2): 143-56, 1974.

### Disposable Tip for Directly Coupled Electrode

In some embodiments herein, the various devices and methods can comprise and/or utilize a disposable tip between an electrode such as described in the section “Electrode Design to Avoid Edge and Corner Effects” (see above) and a patient. Such a disposable tip provides a barrier to patient contamination and couples both the RF energy and the cooling between the electrode and the tissue.

In various conformations, the disposable tip maintains thermal and electrical contact with an electrode while providing a physical barrier to the patient. It will be appreciated that the electrode, used to conduct energy to the patient's tissue, can be in direct contact with the tissue and can be of various shapes, e.g., round, polygonal, etc. Also, the energy from the electrode can be RF energy, e.g., delivered in monopolar or bipolar fashion.

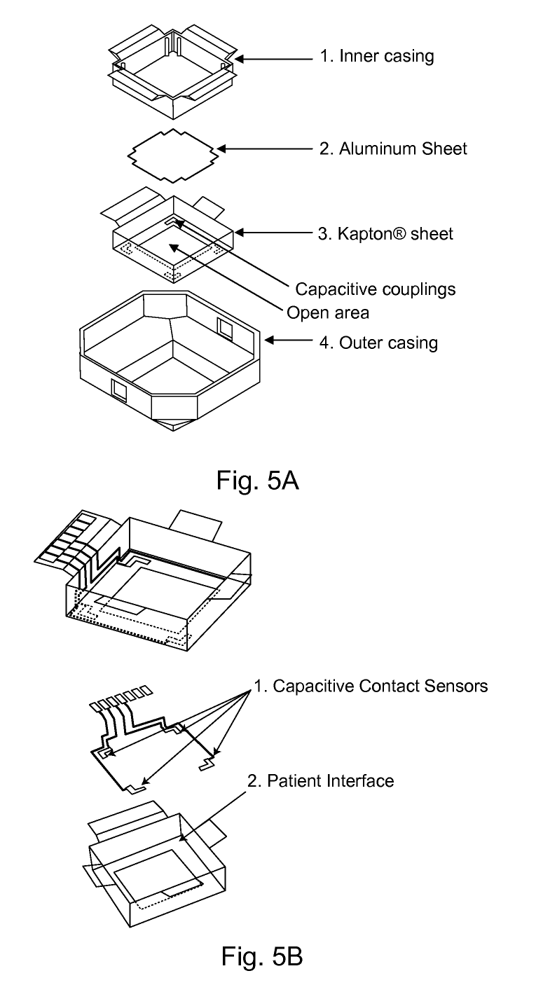

FIGS. 5a-b show an exemplary disposable tip of the invention. As can be seen in the figure, exemplary embodiments of a disposable tip can be comprised of four main components: an inner casing, a metal or metallic sheet (e.g., aluminum), a flex circuit, and an outer casing. In some embodiments, during manufacture, the metal sheet is bonded to the flex circuit. The flex circuit and metal sheet can be folded at the corners, stretched across the bottom of thinner casing and subsequently bonded to the inner casing on the sides. The inner casing can then be inserted and bonded into the outer casing. The inner and outer casings may be made of a polymer, such as Kapton® or other polymer(s). The metal sheet provides a direct coupling to the patient while the outer casing provides a base for capacitive couplings at the corners. It will be appreciated that in various embodiments such components can be comprised of various combinations of plastics, metals, alloys, etc.

In some embodiments the disposable tip also includes an electronic chip used to track usage of the tip. In some such embodiments, the usage tracking prevents the tip from being used past a maximum number of RF energy delivery cycles. In other embodiments, the electrode tip becomes non-functional when its age subsequent to manufacture (“expiration date”) has surpassed a threshold. In this manner the integrity and functionality of the electrode tip are better ensured, by preventing overuse, or use of tips that are past their expiration date. In some embodiments, the information stored in the electronic chip is encrypted, to prevent tampering. In various embodiments, the chip comprises an eprom. Alternatively these parameters and pieces of information can be monitored and controlled from a main console, computer, or computer processor.

Various references useful for the embodiments of such embodiments, as well as optionally for other sections and embodiments herein, include, e.g., Allain, J. C.; Le Lous, M.; Bazin, S.; Bailey, A. J.; and Delaunay, A.: Isometric tension developed during heating of collagenous tissues. Relationships with collagen cross-linking. Biochim Biophys Acta, 533(1): 147-55, 1978; Allain, J. C.; Le Lous, M.; Cohen, S.; Bazin, S.; and Maroteaux, P.: Isometric tensions developed during the hydrothermal swelling of rat skin. Connect Tissue Res, 7(3): 127-33, 1980; Arnoczky, S. P., and Aksan, A.: Thermal modification of connective tissues: basic science considerations and clinical implications. Instr Course Lect, 50: 3-11, 2001; Avitall, B. et al.: Physics and engineering of transcatheter cardiac tissue ablation. J Am Coll Cardiol, 22(3): 921-32, 1993; Avitall, B.; Lafontaine, D.; Rozmus, G.; Adoni, N.; Le, K. M.; Dehnee, A.; and Urbonas, A.: The safety and efficacy of multiple consecutive cryo lesions in canine pulmonary veins-left atrial junction. Heart Rhythm, 1(2): 203-9, 2004; Avitall, B.; Urbonas, A.; Urboniene, D.; Rozmus, G.; and Helms, R.: Linear lesions provide protection from atrial fibrillation induction with rapid atrial pacing. J Cardiovasc Electrophysiol, 13(5): 455-62, 2002; Brodkey, J. S.; Miyazaki, Y.; Ervin, F. R.; and Mark, V. H.: Reversible Heat Lesions With Radiofrequency Current. A Method Of Stereotactic Localization. J Neurosurg, 21: 49-53, 1964; Chen, S. S.; Wright, N. T.; and Humphrey, J. D.: Heat-induced changes in the mechanics of a collagenous tissue: isothermal, isotonic shrinkage. J Biomech Eng, 120(3): 382-8, 1998; Cooper, T. E., and Trezek, G. J.: Correlation of thermal properties of some human tissue with water content. Aerosp Med, 42(1): 24-7, 1971; Cosman, E. R.; Nashold, B. S.; and Ovelman-Levitt, J.: Theoretical aspects of radiofrequency lesions in the dorsal root entry zone. Neurosurgery, 15(6): 945-50, 1984; Daoud, E., and Morady, F.: Catheter ablation of ventricular tachycardia. Curr Opin Cardiol, 10(1): 21-5, 1995; Hayashi, K.; Hecht, P.; Thabit, G., 3rd; Peters, D. M.; Vanderby, R., Jr.; Cooley, A. J.; Fanton, G. S.; Orwin, J. F.; and Markel, M. D.: The biologic response to laser thermal modification in an in vivo sheep model. Clin Orthop Relat Res, (373): 265-76, 2000; Hayashi, K.; Markel, M. D.; Thabit, G., 3rd; Bogdanske, J. J.; and Thielke, R. J.: The effect of nonablative laser energy on joint capsular properties. An in vitro mechanical study using a rabbit model. Am J Sports Med, 23(4): 482-7, 1995; Hayashi, K.; Peters, D. M.; Thabit, G., 3rd; Hecht, P.; Vanderby, R., Jr.; Fanton, G. S.; and Markel, M. D.: The mechanism of joint capsule thermal modification in an in-vitro sheep model. Clin Orthop Relat Res, (370): 236-49, 2000; Hayashi, K.; Thabit, G., 3rd; Bogdanske, J. J.; Mascio, L. N.; and Markel, M. D.: The effect of nonablative laser energy on the ultrastructure of joint capsular collagen. Arthroscopy, 12(4): 474-81, 1996; Lesh, M. D.: Interventional electrophysiology—state-of-the-art 1993. Am Heart J, 126(3 Pt 1): 686-98, 1993; Moraci, A.; Buonaiuto, C.; Punzo, A.; Parlato, C.; and Amalfi, R.: Trigeminal neuralgia treated by percutaneous thermocoagulation. Comparative analysis of percutaneous thermocoagulation and other surgical procedures. Neurochirurgia (Stuttg), 35(2): 48-53, 1992; Saxon, L. A.; Kalman, J. M.; Olgin, J. E.; Scheinman, M. M.; Lee, R. J.; and Lesh, M. D.: Results of radiofrequency catheter ablation for atrial flutter. Am J Cardiol, 77(11): 1014-6, 1996; Seegenschmiedt, M. H., and Sauer, R.: The current role of interstitial thermo-radiotherapy. Strahlenther Onkol, 168(3): 119-40, 1992; Sweet, W. H., and Wepsic, J. G.: Controlled thermocoagulation of trigeminal ganglion and rootlets for differential destruction of pain fibers. 1. Trigeminal neuralgia. J Neurosurg, 40(2): 143-56, 1974.

### Capacitive Contact Sensing Method

The present embodiment pertains to devices and methods that sense the proximity of an electrode to tissue and provide feedback to the practitioner to adjust the electrode placement to maintain effective contact. As will be appreciated, these embodiments can optionally be used in conjunction or along with any of the other embodiments described herein. When RF energy is directly or dielectrically coupled to the tissue of a subject, an electric potential is present in the tissue with respect to the return pad for unipolar RF, or the common electrode in the case of bipolar RF. When an electrode separated by a thin dielectric layer is placed next to the tissue, a voltage can be sensed through the capacitor formed by this electrode, the dielectric material and the tissue (acting as a second electrode). This voltage is then applied to a resistor referenced to the return of the RF signal. The capacitor thus formed has its maximum value when the dielectric is in direct contact with the tissue, and the capacitance decreases as the spacing between its electrodes is increased. In the present embodiments, the sensed voltage across a load resistor is proportional to the applied RF voltage, the frequency, the load sensing resistance, and the capacitance. In many embodiments, the frequency and the sensing load resistance are held constant, and the measurement is normalized with respect to the applied voltage.

In various embodiments, the invention comprises an apparatus comprising a capacitive proximity sensor in which a dielectrically isolated sensing electrode is placed adjacent to the RF delivery electrode on a tissue and is able to sense the applied RF voltage. In some embodiments, the apparatus can have a plurality of sensors placed around the treatment electrode in such a way as to detect the proximity of the perimeter of the RF delivery electrode to the tissue. In some embodiments, the apparatus can have a plurality of sensors placed around the treatment electrode in such a way as to detect the proximity of the perimeter of the RF delivery electrode where the RF delivery electrode is directly coupled to a tissue. Also, in some embodiments, the apparatus can have a plurality of sensors placed around the treatment electrode in such a way as to detect the proximity of the perimeter of the RF delivery electrode where the RF delivery electrode is dielectrically coupled to a tissue. In some embodiments, the apparatus can comprise a disposable tip that maintains thermal and electrical contact with the electrode, while providing a physical barrier to the subject (see above).

In some embodiments, the proximity sensor or sensors are used to provide feedback for the application of the RF energy. For example, in some embodiments sufficient tissue proximity of the RF delivery electrode is required before RF energy is applied. In further embodiments sufficient tissue proximity around the perimeter of the RF delivery electrode is required before RF energy is applied. In yet further embodiments, the strength of the field at different areas on the electrode is varied depending on the measured contact capacitance. In this manner, variations in tissue coupling across the sensor face of the RF delivery electrode may be compensated, resulting in a more uniform application of the RF energy.

In some embodiments, the invention comprises methods for detecting proximity of the tip to the tissue through capacitive conduct. In some such methods, there is set of sensing electrodes that are dielectrically isolated from the tissue, wherein the RF voltage is sensed through the electrode capacitively. In some embodiments, the RF voltage sensed is proportional to the capacitance of the electrodes. In such embodiments, the sensing capacitors terminals can be the electrode on one side and the tissue on the other, and the sensing capacitance can decrease with separation between the tip and the tissue.

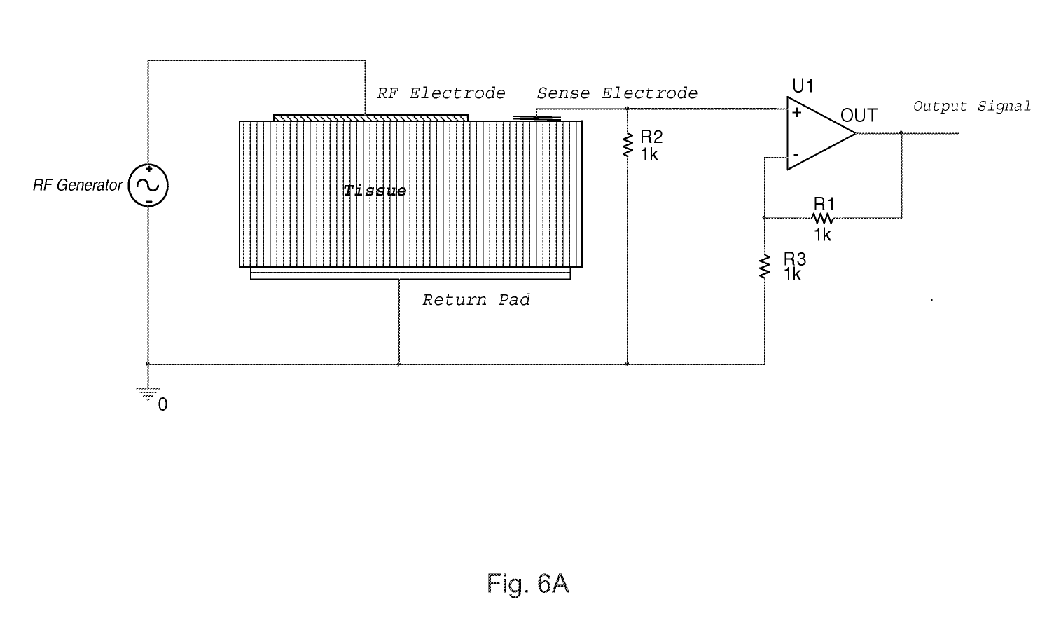

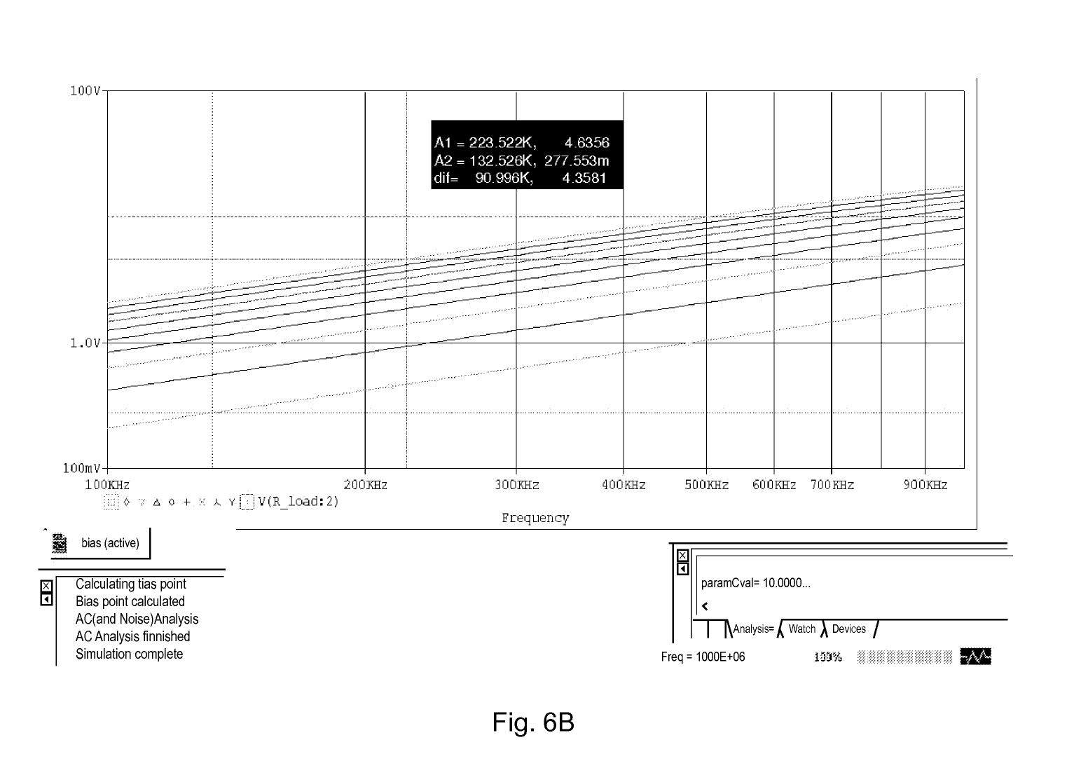

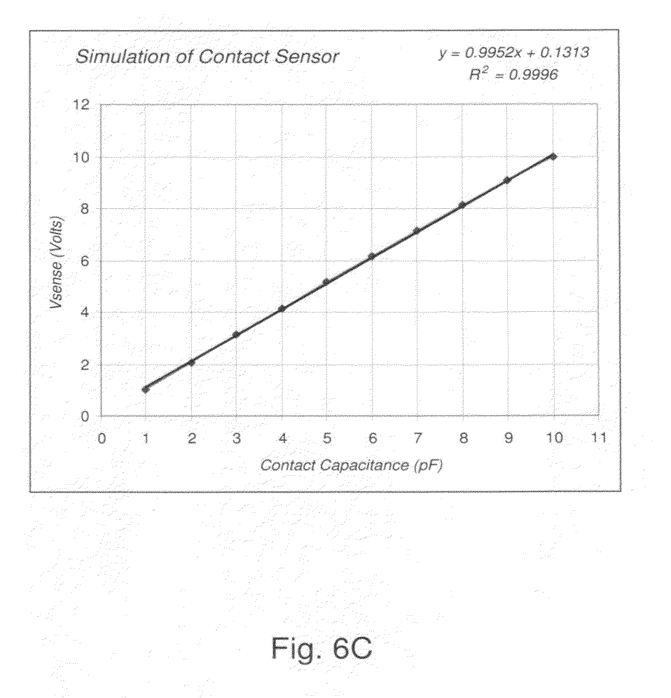

FIG. 6a shows a circuit diagram representation of tissue in contact with a treatment electrode and a sensor electrode. The sensor electrode is dielectrically isolated from the tissue and acts as a capacitor, with one capacitor electrode being the electrode conductor and the other capacitor electrode being the tissue itself. Resistor R2 returns the current flowing through the sensor capacitance to the common of the RF generator and develops a voltage across itself. This voltage is buffered and/or amplified by the operational amplifier U1.

The fraction of the RF voltage present at a sense point, V_sense, across the resistor R2 can be modeled according to:

V_sense=V₀*2π*f*R2*C/(1+(2π*f*R2)2)

where V₀ is proportional to the applied RF Voltage, and, f, is the frequency of the RF energy applied. With component values (C=5×10̂-12 Farads, f=500×10̂3 Hz, R2=1000 Ohms 2π*f*R2*C=2.5×10̂-3 which is much smaller than 1), the equation therefore reduces to:
V_sense=V₀*2π*f*R2*C
From this equation it can be seen that the sensed voltage is directly proportional to the capacitance between the tissue and sensing electrode.

A simulation of the circuit shown in FIG. 6a is summarized in FIG. 6b. In FIG. 6b, the horizontal axis is frequency, the vertical axis is Vsense. The parameter being varied during the simulation is the sense resistor (R2 in the above equation). The set of graphs shows the Vsense increasing as a function of frequency and R2 (in this simulation C is constant, but a similar simulation with R2 constant and C changing could also be done. A plot of the sensing voltage as a function of contact capacitance (FIG. 6c) confirms their linear relationship. As noted above a second parametric simulation with C changing was done, and the plot in FIG. 6c shows how Vsense varies as a function of contact capacitance.

In this embodiment as well, it will be appreciated that the methods and devices of the embodiment can also comprise additional components, e.g., computer or computer processing components to monitor the capacitive contact sensing, etc. Additionally, the capacitive contact sensing device/methods can be used in conjunction with or along with any of the other embodiments herein, e.g., the tissue/collagen monitoring embodiments, the temperature controlled electrode designs, etc.

### Magnetic Sensing of Fluidics Connector

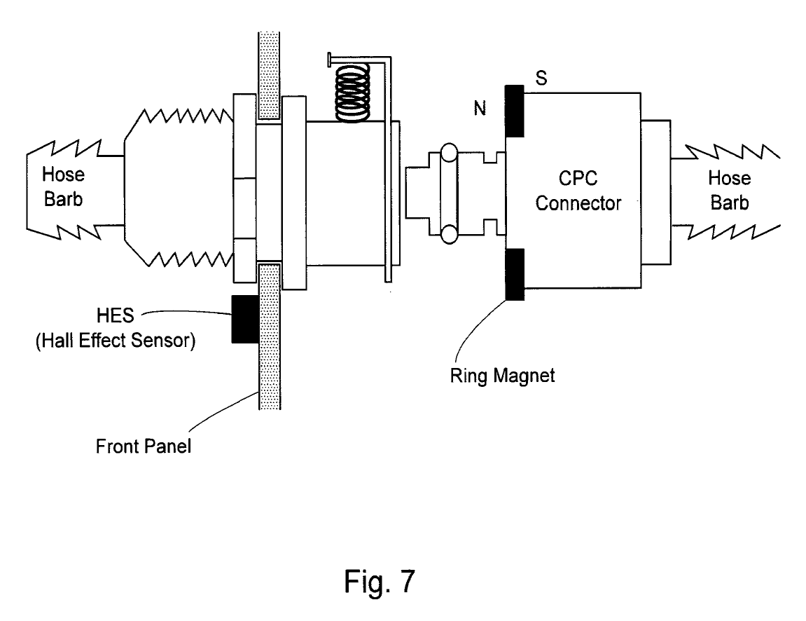

In various embodiments herein, the devices can optionally comprise one or more magnetic sensing methods to, e.g., determine whether fluidic components such as a plug and its mate (see, e.g., FIG. 7a) are properly attached and/or oriented. It will be appreciated, however, that the magnetic sensing components herein are widely applicable and can be used in conjunction with myriad other systems/components in addition to those described herein.

In many common devices and systems, typical fluidics connectors and non-electrical connectors do not provide a convenient low cost method by which it can be automatically detected whether they are properly connected. The current embodiment provides methods and exemplary components to conveniently determine attachment/orientation of fluidic components. More specifically, in medical devices, and in scientific and industrial instrumentation in general, there is a need for automatically detecting whether or not a non-electrical connection has been made. Some complex approaches such as RFID are used in various systems, but such approaches are costly for the purpose of simply detecting the presence or absence of the connector. Furthermore, incorporating such electrical contacts within fluidics connectors adds unnecessary complexity and cost as well.

The present embodiment uses magnetic sensors (such as, but not limited to, Hall Effect Sensors which vary their output voltage in response to changes in a magnetic field) in conjunction with a permanent magnet to sense the proximity of a fluidics connector. The present approach allows non-electrical connectors to be free of wires and active components (advantages that are important in the manufacturing process), and yet provide an electrical signal that signifies the presence of the connector. The present embodiment utilizes magnetic sensors to detect the presence of an appropriately magnetized mating connector. In typical embodiments, no wires or active circuitry need be added to the fluidics connector to allow for its presence to be detected: only a magnet is required.

In one exemplary embodiment, a ring magnet can be polarized axially (i.e. its poles are on the faces of the ring), and slipped on the body of a plug proximal to the tip of a connector pair. A Hall Effect Sensor (IES) can be located nearby, e.g., behind a panel near to where the mating connector (the receptacle) is mounted. When the plug is mated with receptacle, the magnetic field of the permanent magnet activates the HES and the connection is detected. The HES can differentiate between the north and south poles of the ring magnet, and therefore can distinguish between two identical connectors equipped with oppositely polarized magnets. See, e.g., FIG. 7.

In some embodiments, the invention comprises a fluidics connector with a permanent ring magnet attached to the removable half of the connector so as to activate a magnetic sensor in the proximity of the other half of the connector. In such fluidics connectors, the removable half of the connector can be free to rotate around its axis and still maintain detectability by the sensor (e.g., due to axially polarized ring magnet). Also, in some embodiments, the ring magnet can be insert-molded to the removable half or be attached to the removable half, post-manufacture. Also, in some embodiments, the magnet can be polarized axially. Also, the magnet's north pole can be facing the mating connector and/or the magnet's south pole can be facing the mating connector. In some embodiments, a magnetic sensor can be placed behind a panel that holds the connector without modifications to the connector itself in such a way as to detect the proximity of the mating connector, while in other embodiments, a magnetic sensor can be placed behind a panel holding the connector in such a way as to detect the proximity of the mating connector.

In some embodiments, a signal, indicating proximity of a connector, is used as a way of detecting a fault state of the instrument. For example, the instrument may be prevented from operating when certain connectors are not in place. In some embodiments, the instrument user may be alerted to the absence of the connector through an indicator or message on a display.

Such connection detectors can be used with various of the other embodiments herein, e.g., to determine if proper fluidic connections exist for TEC aspects of the electrodes, etc. The connection detectors can optionally be operably connected to one or more computer or computer processing component and/or can be operably connected to components in the temperature controlled electrode aspects, etc.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

1. A sensor system for monitoring collagen content and/or collagen status in one or more tissues, the system comprising:
a signal generator which generates a signal to be transmitted to one or more tissues, which signal can be related to collagen content and/or collagen status in the one or more tissues;
a connector operably connected to the signal generator, which connector carries the signal from the signal generator to the one or more tissues and which receives one or more signals back from the one or more tissues; and,
a monitor which is operably connected to the connector, and which generates an output derived from the one or more signals back from the one or more tissues which corresponds to the collagen content and/or collagen status in the one or more tissues.

2.-33. (canceled)

34. A method of measuring collagen content and/or collagen status in one or more tissues in a subject, the method comprising:
providing a signal generator which generates a signal to be transmitted to one or more tissues, which signal can be corresponded to collagen content and/or collagen status in the one or more tissues;
providing a connector operably connected to the signal generator, which connector carries the signal from the signal generator to the one or more tissues and which receives one or more signals back from the one or more tissues;
providing a monitor which is operably connected to the connector and/or signal generator, and which generates an output derived from the one or more signals back from the one or more tissues which corresponds to the collagen content and/or collagen status in the one or more tissues;
inserting at least a portion of signal generator and/or the connector into a cavity of the subject such that at least a portion of the signal generator and/or the connector is adjacent to a collagen containing tissue;
generating a signal, transmitting it to the tissue, and receiving one or more return signals from the tissue;
conveying the return signal to the monitor and/or to a computer or computer processor wherein the return signal provides information corresponding to collagen content and/or collagen status in the tissue to a user.

35.-54. (canceled)

55. A system or device for monitoring a change in one or more tissular structures, the system or device comprising:
an AC potential source component;
a detection/measuring component; and,
a computer component programmed to:
control the AC potential;
deliver a first AC potential to the analyzed tissue;
direct the detection/measurement component to measure a first permittivity from the one or more tissular structures;
deliver a second AC potential to the analyzed tissue;
direct the detection/measurement component to measure a second permittivity from the one or more tissular structures; and,
compare the first permittivity value and the second permittivity value, thereby monitoring the changes in the one or more collagen structures.

56.-134. (canceled)
